(19) United States
(12) Patent Application Publication
(10) Pub. No.: US 2008/0064876 A1
(43) Pub. Date: Mar. 13, 2008

(54) Process for the preparation of substituted 2-(2,6-dioxopiperidin-3-yl)isoindole-1,3-dione

(76) Inventors: George W. Muller, Bridgewater, NJ (US); Manohar Saindane, Monmouth Junction, NJ (US); Chuansheng Ge, Belle Meade, NJ (US); Mohit Atul Kothare, Green Brook, NJ (US); Louise Michelle Cameron, East Windsor, NJ (US); Mark Edward Rogers, Bordentown, NJ (US)

Correspondence Address: JONES DAY, 222 EAST 41ST ST, NEW YORK, NY, 10017, US

(21) Appl. No.: 11/803,807

(22) Filed: May 15, 2007

Related U.S. Application Data
(60) Provisional application No. 60/800,708, filed on May 16, 2006.

(51) Int. Cl. C07D 401/02

(52) U.S. Cl. 546/201

(57) ABSTRACT

The present invention provides processes for the preparation of substituted 2-(2,6-dioxopiperidin-3-yl)isoindole-1,3-diones which are useful, for example, for preventing or treating diseases or conditions related to an abnormally high level or activity of TNF-α. The invention can provide cost-effective and efficient processes for the commercial production of substituted 2-(2,6-dioxopiperidin-3-yl)isoindole-1,3-diones, including, but not limited to, 4-[(N,N-dimethylhydrazono)methyl]-2-(2,6-dioxopiperidin-3-yl)isoindole-1,3-dione, 4-aminomethyl-2-(2,6-dioxopiperidin-3-yl)isoindole-1,3-dione, and 4-[(acylamino)methyl]-2-(2,6-dioxopiperidin-3-yl)isoindole-1,3-diones.

This application claims the benefit of U.S. provisional application No. 60/800,708, filed May 16, 2006, the content of which is incorporated by reference herein in its entirety.

### 1. FIELD OF THE INVENTION

The present invention provides processes for the preparation of compounds useful for reducing levels or activity of tumor necrosis factor α in mammals. More specifically, the invention provides processes for the preparation of substituted 2-(2,6-dioxopiperidin-3-yl)isoindole-1,3-dione. In particular embodiments, the invention provides processes useful for the preparation of 4-[(N,N-dimethylhydrazono)methyl]-2-(2,6-dioxopiperidin-3-yl)isoindole-1,3-dione, 4-aminomethyl-2-(2,6-dioxopiperidin-3-yl)isoindole-1,3-dione and 4-[(acylamino)methyl]-2-(2,6-dioxopiperidin-3-yl)isoindole-1,3-diones.

### 2. BACKGROUND OF THE INVENTION

Excessive or unregulated production of tumor necrosis factor α or TNF-α, has been implicated in a number of disease conditions. These include endotoxemia and/or toxic shock syndrome (Tracey et al., Nature 330, 662-664 (1987) and Hinshaw et al., Circ. Shock 30, 279-292 (1990)), cachexia (Dezube et al., Lancet 335 (8690), 662 (1990)), and Adult Respiratory Distress Syndrome (Millar et al., Lancet 2 (8665), 712-714 (1989)). Certain substituted 2-(2,6-dioxopiperidin-3-yl)-1-oxoisoindolines have been shown to reduce levels of TNFα (International Publication No. WO 98/03502, incorporated herein by reference in its entirety).

A substituted isoindole-1,3-dione that has demonstrated particular therapeutic promise is 2-(2,6-dioxopiperidin-3-yl)isoindole-1,3-dione (THALOMID™). These compounds have been shown to be or one believed to be useful in treating and preventing a wide range of diseases and conditions including, but not limited to, inflammatory diseases, autoimmune diseases, and cancers.

Existing methods for synthesizing substituted 2-(2,6-dioxopiperidin-3-yl)isoindole-1,3-diones are described in U.S. Patent Application Publication No. 2003/0096841, which is incorporated herein by reference in its entirety. While these methods are enabling and useful for preparing substituted 2-(2,6-dioxopiperidin-3-yl)isoindole-1,3-diones, alternative methods for the preparation of substituted 2-(2,6-dioxopiperidin-3-yl)isoindole-1,3-diones, particularly for manufacturing scale production, are desirable.

Citation of any reference in Section 2 of this application is not to be construed as an admission that such reference is prior art to the present application.

### 3. SUMMARY OF THE INVENTION

The present invention provides cost-effective and efficient processes for the preparation of substituted 2-(2,6-dioxopiperidin-3-yl)isoindole-1,3-diones. In some embodiments, the invention provides processes for preparing a substituted 2-(2,6-dioxopiperidin-3-yl)isoindole-1,3-dione comprising the steps of:

(1) reacting maleic anhydride with a 2-substituted furan to form a substituted isobenzofuran-1,3-dione; and

(2) forming a substituted 2-(2,6-dioxopiperidin-3-yl)isoindole-1,3-dione by reacting the substituted isobenzofuran-1,3-dione with a primary amine having the formula: 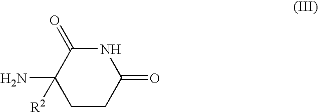
where R² is H, F, benzyl, (C₁-C₈)alkyl, (C₂-C₈)alkenyl, or (C₂-C₈)alkynyl.

In one aspect, the invention provides a process for preparing a compound of Formula (I): 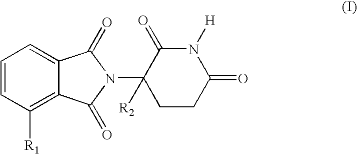
or a pharmaceutically acceptable salt, solvate including a hydrate or polymorph thereof, which comprises the steps of:

(1) reacting a furan of Formula (II): 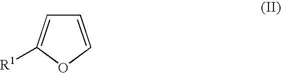
with maleic anhydride to form a compound of Formula (IV):

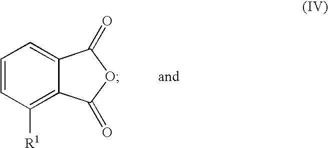

(2) reacting the compound of Formula (IV) with a primary amine having the formula: 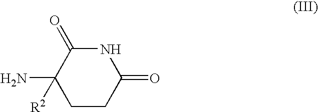
or a salt thereof to form the compound of Formula (I); wherein:

R¹ is —(CH₂)ₙ—NH—R′;

R² is H, F, benzyl, (C₁-C₈)alkyl, (C₂-C₈)alkenyl, or (C₂-C₈)alkynyl;

R′ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl)-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group; and

n is 0 or 1.

In another aspect, the invention provides a process for preparing a compound of Formula (IV) or a pharmaceutically acceptable salt, solvate including a hydrate or polymorph thereof, which comprises the step of reacting a furan of Formula (II) with maleic anhydride in ethyl acetate with the presence of an organic acid.

In another aspect, the invention provides a process for preparing a compound of Formula (I) or a pharmaceutically acceptable salt, solvate including a hydrate or polymorph thereof, which comprises the step of reacting a compound of Formula (IV) with a primary amine of Formula (III) or a salt thereof in the presence of a mixture of acetic acid and imidazole.

In another aspect, the invention provides a process for preparing a compound of Formula (I) or a pharmaceutically acceptable salt, solvate including a hydrate or polymorph thereof, which comprises the step of reacting a furan of Formula (II) with a heterocyclic compound of Formula (V): 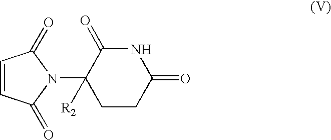
wherein:

R² is H, F, benzyl, (C₁-C₈)alkyl, (C₂-C₈)alkenyl, or (C₂-C₈)alkynyl.

In one embodiment, the invention provides a process for preparing a compound of Formula (I) or a pharmaceutically acceptable salt, solvate including a hydrate or polymorph thereof, which comprises the step of reacting a furan of Formula (II) with a heterocyclic compound of Formula (V) in ethyl acetate with the presence of an organic acid.

In another aspect, the invention provides a process for preparing 4-[(N,N-dimethylhydrazono)methyl]-2-(2,6-dioxopiperidin-3-yl)isoindole-1,3-dione having the formula: 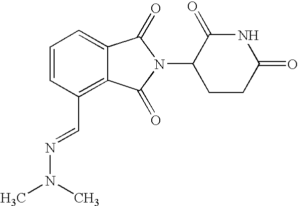
or a pharmaceutically acceptable salt, solvate including a hydrate or polymorph thereof, which comprises the steps of:

(1) reacting maleic anhydride with 2-furaldehyde dimethylhydrazone having the formula: 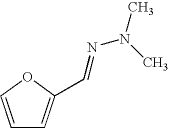
in a first solvent at a first temperature above room temperature to form an isobenzofuran having the formula:

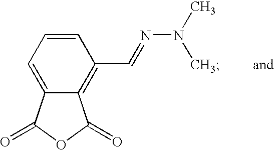

(2) reacting the isobenzofuran with 3-aminopiperidine-2,6-dione hydrochloride in a second solvent at a second temperature above room temperature to form the 4-[(N,N-dimethylhydrazono)methyl]-2-(2,6-dioxopiperidin-3-yl)isoindole-1,3-dione.

In one embodiment, the first step occurs in the presence of trifluoroacetic acid. In a further embodiment, the first solvent is ethyl acetate. In a further embodiment, the first temperature is between 45° C. and 55° C. In a further embodiment, the second step occurs in the presence of a mixture of acetic acid and imidazole. In a further embodiment, the second solvent is acetonitrile. In a further embodiment, the second temperature is between 75° C. and 85° C.

In a further embodiment, the —CH═N—N(CH₃)₂ group of the 4-[(N,N-dimethylhydrazono)methyl]-2-(2,6-dioxopiperidin-3-yl)isoindole-1,3-dione is reduced to a —CH₂NH₂ group by hydrogen in the presence of 10% Pd/C and methanesulfonic acid to form a mesylate salt of 4-aminomethyl-2-(2,6-dioxopiperidin-3-yl)isoindole-1,3-dione. In a further embodiment, the mesylate salt is converted into a hydrochloride salt by reacting the mesylate salt with 12N hydrochloric acid.

In a further embodiment, the —CH₂—NH₂ group of the 4-aminomethyl-2-(2,6-dioxopiperidin-3-yl)isoindole-1,3-dione hydrochloride reacts with cyclopropanecarbonyl chloride in the presence of diisopropylethylamine in acetonitrile at a temperature between 0° C. and 20° C. to form 4-[(cyclopropanecarbonylamino)methyl]-2-(2,6-dioxopiperidin-3-yl)isoindole-1,3-dione.

The processes of the present invention offer several advantages over conventional methods for the preparation of substituted 2-(2,6-dioxopiperidin-3-yl)isoindole-1,3-diones. First, less expensive starting materials and reagents can be employed. For instance, maleic anhydride and substituted furans can be relatively inexpensive. Second, in some embodiments, there can be fewer steps in the processes of this invention. Third, the stereochemistry of the product can be controlled partially by controlling the stereochemistry of one of the starting material, i.e., 3-aminopiperidine-2,6-dione and 4-alkyl-3-aminopiperidine-2,6-dione. Other advantages are also contemplated.

### 4. DETAILED DESCRIPTION OF THE INVENTION

4.1 Terminology

As used herein and unless otherwise indicated, the term “halo”, “halogen” or the like means —F, —Cl, —Br or —I.

As used herein and unless otherwise indicated, the term “alkyl” or “alkyl group” means a univalent group having the general formula CₙH₂ₙ₊₁ derived from removing a hydrogen atom from a saturated, unbranched or branched aliphatic hydrocarbon, where n is an integer, preferably between 1 and 20, more preferably between 1 and 8. Examples of alkyl groups include, but are not limited to, (C₁-C₈)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl and octyl. Longer alkyl groups include nonyl and decyl groups. An alkyl group can be unsubstituted or substituted with one or more suitable substituents. Furthermore, the alkyl group can be branched or unbranched.

As used herein and unless otherwise indicated, the term “methylene” means a divalent —CH₂— group.

As used herein and unless otherwise indicated, the term “carbonyl” means a divalent —C(═O)— group.

As used herein and unless otherwise indicated, the term “heteroalkyl” or “heteroalkyl group” means a univalent group derived from an alkyl group with at least one of the methylene group is replaced by a heteroatom or a hetero-group such as O, S, or NR where R is H or an organic group.

As used herein and unless otherwise indicated, the term “organic group” means a group containing at least a carbon atom. Examples of the organic group include, but are not limited to, alkyl, heteroalkyl, alkenyl, alkynyl, carboxyl, acyl, aryl, heteroaryl, cycloalkyl, and heterocycloalkyl.

As used herein and unless otherwise indicated, the term “cycloalkyl” or “cycloalkyl group” means a univalent group derived from a cycloalkane by removal of a hydrogen atom from a non-aromatic, monocyclic or polycyclic ring comprising carbon and hydrogen atoms. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes and (C₃-C₇)cycloalkenyl groups, such as cyclopropenyl, cyclobutenyl, cyclopentenyl, cyclohexenyl, and cycloheptenyl, and unsaturated cyclic and bicyclic terpenes. A cycloalkyl group can be unsubstituted or substituted by one or two suitable substituents. Furthermore, the cycloalkyl group can be monocyclic or polycyclic.

As used herein and unless otherwise indicated, the term “alkoxy” or “alkoxy group” means an alkyl group that is linked to another group via an oxygen atom (i.e., —O-alkyl). An alkoxy group can be unsubstituted or substituted with one or more suitable substituents. Examples of alkoxy groups include, but are not limited to, (C₁-C₆)alkoxy groups, such as —O-methyl, —O-ethyl, —O-propyl, —O-isopropyl, —O-2-methyl-1-propyl, —O-2-methyl-2-propyl, —O-2-methyl-1-butyl, —O-3-methyl-1-butyl, —O-2-methyl-3-butyl, —O-2,2-dimethyl-1-propyl, —O-2-methyl-1-pentyl, 3-O-methyl-1-pentyl, —O-4-methyl-1-pentyl, —O-2-methyl-2-pentyl, —O-3-methyl-2-pentyl, —O-4-methyl-2-pentyl, —O-2,2-dimethyl-1-butyl, —O-3,3-dimethyl-1-butyl, —O-2-ethyl-1-butyl, —O-butyl, —O-isobutyl, —O-t-butyl, —O-pentyl, —O-isopentyl, —O-neopentyl and —O-hexyl. An alkoxy group can be unsubstituted or substituted with one or two suitable substituents. Preferably, the alkyl chain of an alkyloxy group is from 1 to 8 carbon atoms in length, referred to herein as “(C₁-C₈)alkoxy”.

As used herein and unless otherwise indicated, the term “heterocycloalkyl” or “heterocycloalkyl group” means a univalent group derived from a monocyclic or polycyclic heterocycloalkane by removal of a hydrogen atom from a ring carbon atom. Non-limiting examples of the heterocycloalkyl group include oxirane, thiirane, aziridine, oxetane, thietane, azetidine, pyrrolidine, tetrahydrothiophene, tetrahydrofuran, 2-pyrrolidinone, 2,5-pyrrolidinedione, dihydro-2(3H)-furanone, dihydro-2,5-furandione, dihydro-2(3H)-thiophenone, 3-aminodihydro-2(3H)-thiophenone, piperidine, 2-piperidinone, 2,6-piperidinedione, tetrahydro-2H-pyran, tetrahydro-2H-pyran-2-one, dihydro-2H-pyran-2,6(3H)-dione, and tetrahydro-4H-thiopyran-4-one. A heterocycloalkyl group can be unsubstituted or substituted with one or more suitable substituents. Furthermore, the heterocycloalkyl group can be monocyclic or polycyclic.

As used herein and unless otherwise indicated, the term “aryl” or “aryl group” means an organic radical derived from a monocyclic or polycyclic aromatic hydrocarbon by removing a hydrogen atom. Non-limiting examples of the aryl group include phenyl, naphthyl, benzyl, or tolanyl group, sexiphenylene, phenanthrenyl, anthracenyl, coronenyl, and tolanylphenyl. An aryl group can be unsubstituted or substituted with one or more suitable substituents. Furthermore, the aryl group can be monocyclic or polycyclic.

As used herein and unless otherwise indicated, the term “heteroaryl” or “heteroaryl group” means an organic radical derived from a monocyclic or polycyclic aromatic heterocycle by removing a hydrogen atom. Non-limiting examples of the heteroaryl group include furyl, thienyl, pyrrolyl, indolyl, indolizinyl, isoindolyl, pyrazolyl, imidazolyl, thiazolyl, thiadiazolyl, benzothiazolyl, 1,2,4-triazolyl, 1,2,3-triazolyl, indazolyl, benzotriazolyl, benzimidazolyl, indazolyl carbazolyl, carbolinyl, benzofuranyl, isobenzofuranyl benzothiophenyl, dibenzofuranyl, dibenzothiophenyl, isothiazolyl, isoxazolyl, pyridyl, purinyl, pyridazinyl, pyrimidinyl, pyrazinyl, triazinyl, tetrazinyl, petazinyl, quinolinyl, isoquinolinyl, perimidinyl, cinnolinyl, phthalazinyl, quinazolinyl, quinoxalinyl, naphthyridinyl, acridinyl, phenanthridinyl, phenanthrolinyl, anthyridinyl, purinyl, pteridinyl, alloxazinyl, phenazinyl, phenothiazinyl, phenoxazinyl, phenoxathiinyl, dibenzo(1,4)dioxinyl, and thianthrenyl. A heteroaryl group can be unsubstituted or substituted with one or more suitable substituents. Furthermore, the heteroaryl group can be monocyclic or polycyclic.

As used herein and unless otherwise indicated, the term “alkenyl” or “alkenyl group” means a monovalent, unbranched or branched hydrocarbon chain having one or more double bonds therein. The double bond of an alkenyl group can be unconjugated or conjugated to another unsaturated group. Suitable alkenyl groups include, but are not limited to (C₂-C₈)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents. Furthermore, the alkenyl group can be branched or unbranched.

As used herein and unless otherwise indicated, the term “alkynyl” or “alkynyl group” means monovalent, unbranched or branched hydrocarbon chain having one or more triple bonds therein. The triple bond of an alkynyl group can be unconjugated or conjugated to another unsaturated group. Suitable alkynyl groups include, but are not limited to, (C₂-C₈)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted with one or two suitable substituents. Furthermore, the alkynyl group can be branched or unbranched.

As used herein and unless otherwise indicated, the term “aryloxy” or “aryloxy group” means an O-aryl group, wherein aryl is as defined above. An aryloxy group can be unsubstituted or substituted with one or two suitable substituents. Preferably, the aryl ring of an aryloxy group is a monocyclic ring, wherein the ring comprises 6 carbon atoms, referred to herein as “(C₆)aryloxy”.

As used herein and unless otherwise indicated, the term “alkoxycarbonyl” or “alkoxycarbonyl group” means a monovalent group of the formula C(═O)-alkoxy. Preferably, the hydrocarbon chain of an alkoxycarbonyl group is from 1 to 8 carbon atoms in length, referred to herein as a “lower alkoxycarbonyl” group.

As used herein and unless otherwise indicated, the term “alkylsulfanyl” or “alkylsulfanyl group” means a monovalent group of the formula —S-alkyl. Preferably, the hydrocarbon chain of an alkylsulfanyl group is from 1 to 8 carbon atoms in length, referred to herein as a “lower alkylsulfanyl” group.

As used herein and unless otherwise indicated, the term “acyloxy” or “acyloxy group” means a monovalent group of the formula —O—C(═O)-alkyl or —O—C(═O)-aryl.

As used herein and unless otherwise indicated, the term “acyl” or “acyl group” means a monovalent group of the formula —C(═O)H, —C(═O)-alkyl or —C(═O)-aryl.

As used herein and unless otherwise indicated, the term “amino” or “amino group” means a monovalent group of the formula —NH₂, —NH(alkyl), —NH(aryl), —N(alkyl)₂, —N(aryl)₂ or —N(alkyl)(aryl).

As used herein and unless otherwise indicated, the term “amido” or “amido group” means a monovalent group of the formula —C(═O)NH₂, —C(═O)NH(alkyl), —C(═O)NH(aryl), —C(═O)N(alkyl)₂, —C(═O)N(aryl)₂ or —C(═O)N(alkyl)(aryl).

As used herein and unless otherwise indicated, the term “acylamino” or “acylamino group” means a monovalent group of the formula —NH—C(═O)-alkyl, —N(alkyl)-C(═O)-alkyl, —NH—C(═O)-aryl, —N(alkyl)-C(═O)-aryl, —N(aryl)-C(═O)-alkyl or —N(aryl)-C(═O)-aryl.

As used herein and unless otherwise indicated, the term “substituted” as used to describe a compound or chemical moiety means that at least one hydrogen atom of that compound or chemical moiety is replaced with a second chemical moiety. The second chemical moiety can be any desired substituent that does not adversely affect the desired activity of the compound. Examples of substituents are those found in the exemplary compounds and embodiments disclosed herein, as well as halogen; alkyl; heteroalkyl; alkenyl; alkynyl; aryl, heteroaryl, hydroxyl; alkoxyl; amino; nitro; thiol; thioether; imine; cyano; amido; phosphonato; phosphine; carboxyl; thiocarbonyl; sulfonyl; sulfonamide; ketone; aldehyde; ester; oxo; haloalkyl (e.g., trifluoromethyl); carbocyclic cycloalkyl, which can be monocyclic or fused or non-fused polycyclic (e.g., cyclopropyl, cyclobutyl, cyclopentyl or cyclohexyl) or a heterocycloalkyl, which can be monocyclic or fused or non-fused polycyclic (e.g., pyrrolidinyl, piperidinyl, piperazinyl, morpholinyl or thiazinyl); carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl or benzofuranyl); amino (primary, secondary or tertiary); o-lower alkyl; o-aryl, aryl; aryl-lower alkyl; —CO₂CH₃; —CONH₂; —OCH₂CONH₂; —NH₂; —SO₂NH₂; —OCHF₂; —CF₃; —OCF₃; —NH(alkyl); —N(alkyl)₂; —NH(aryl); —N(alkyl)(aryl); —N(aryl)₂; —CHO; —CO(alkyl); —CO(aryl); —CO₂(alkyl); and —CO₂(aryl); and such moieties can also be optionally substituted by a fused-ring structure or bridge, for example —OCH₂O—. These substituents can optionally be further substituted with a substituent selected from such groups. All chemical groups disclosed herein can be substituted, unless it is specified otherwise.

As used herein and unless otherwise indicated, a composition that is “substantially free” of a compound means that the composition contains less than about 20% by weight, more preferably less than about 10% by weight, even more preferably less than about 5% by weight, and most preferably less than about 3% by weight of the compound.

As used herein and unless otherwise indicated, the term “stereochemically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “enantiomerically pure” means a stereomerically pure composition of a compound having one chiral center.

As used herein and unless otherwise indicated, the term “racemic” or “racemate” means about 50% of one enantiomer and about 50% of the corresponding enantiomer relative to all chiral centers in the molecule. The invention encompasses all enantiomerically pure, enantiomerically enriched, diastereomerically pure, diastereomerically enriched, and racemic mixtures of the compounds of the invention.

As used herein and unless otherwise indicated, the term “process(es) of the invention” refers to the methods disclosed herein which are useful for preparing a compound of the invention. Modifications to the methods disclosed herein (e.g., starting materials, reagents, protecting groups, solvents, temperatures, reaction times, purification) are also encompassed by the present invention.

As used herein and unless otherwise indicated, the term “adding”, “reacting” or the like means contacting one reactant, reagent, solvent, catalyst, reactive group or the like with another reactant, reagent, solvent, catalyst, reactive group or the like. Reactants, reagents, solvents, catalysts, reactive group or the like can be added individually, simultaneously or separately and can be added in any order. They can be added in the presence or absence of heat and can optionally be added under an inert atmosphere. “Reacting” can refer to in situ formation or intramolecular reaction where the reactive groups are in the same molecule.

As used herein and unless otherwise indicated, a reaction that is “substantially complete” or is driven to “substantial completion” means that the reaction contains more than about 80% by percent yield, more preferably more than about 90% by percent yield, even more preferably more than about 95% by percent yield, and most preferably more than about 97% by percent yield of the desired product.

As used herein and unless otherwise indicated, the term “pharmaceutically acceptable salt” includes, but is not limited to, salts of acidic or basic groups that may be present in the compounds of the invention. Compounds of the invention that are basic in nature are capable of forming a wide variety of salts with various inorganic and organic acids. The acids that may be used to prepare pharmaceutically acceptable salts of such basic compounds are those that form salts comprising pharmacologically acceptable anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate. Compounds of the invention that include an amino group also can form pharmaceutically acceptable salts with various amino acids, in addition to the acids mentioned above. Compounds of the invention that are acidic in nature are capable of forming base salts with various pharmacologically acceptable cations. Non-limiting examples of such salts include alkali metal or alkaline earth metal salts and, particularly, calcium, magnesium, sodium, lithium, zinc, potassium, and iron salts.

As used herein and unless otherwise indicated, the term “hydrate” means a compound of the present invention or a salt thereof, that further includes a stoichiometric or non-stoichiometeric amount of water bound by non-covalent intermolecular forces.

As used herein and unless otherwise indicated, the term “solvate” means a solvate formed from the association of one or more solvent molecules to a compound of the present invention. The term “solvate” includes hydrates (e.g., mono-hydrate, dihydrate, trihydrate, tetrahydrate, and the like).

As used herein and unless otherwise indicated, the term “polymorph” means solid crystalline forms of a compound of the present invention or complex thereof. Different polymorphs of the same compound can exhibit different physical, chemical and/or spectroscopic properties.

As used herein and unless otherwise indicated, the phrase “diseases or conditions related to an abnormally high level or activity of TNF-α” means diseases or conditions that would not arise, endure or cause symptoms if the level or activity of TNF-α were lower, or diseases or conditions that can be prevented or treated by a lowering of TNF-α level or activity.

Acronyms or symbols for groups or reagents have the following definition: HPLC=high performance liquid chromatography, TFA=trifluoroacetic acid; THF=tetrahydrofuran; EtOAc=ethyl acetate; AcOH=acetic acid; CH₃CN=acetonitrile; NMP=N-methyl pyrrolidinone, MsOH=methanesulfonic acid, DMF=dimethyl formamide, DMSO=dimethyl sulfoxide, and DBU=1,8-diazabicyclo[5.4.0]undec-7-ene.

If there is a discrepancy between a depicted structure and a name given that structure, the depicted structure is to be accorded more weight. Furthermore, if the stereochemistry of a structure or a portion thereof is not indicated, e.g., with bold or dashed lines, the structure or portion thereof is to be interpreted as encompassing all stereoisomers of it.

The invention can be understood more fully by reference to the following detailed description and illustrative examples, which are intended to exemplify non-limiting embodiments of the invention.

4.2 Processes of the Invention

The present invention provides processes for the preparation of substituted 2-(2,6-dioxopiperidin-3-yl)isoindole-1,3-diones. In general, the processes of the present invention are to encompass cost-effective and efficient means for the large scale or commercial production of substituted 2-(2,6-dioxopiperidin-3-yl)isoindole-1,3-diones.

In one embodiment, the invention provides a process for preparing a substituted 2-(2,6-dioxopiperidin-3-yl)isoindole-1,3-dione comprising the steps of:

(1) reacting maleic anhydride with a 2-substituted or unsubstituted furan to form a corresponding substituted or unsubstituted isobenzofuran-1,3-dione; and

(2) reacting the substituted or unsubstituted isobenzofuran-1,3-dione with a primary amine of Formula (III) above, such as 3-aminopiperidine-2,6-dione, or a salt thereof.

The 2-substituted furan can comprise at the 2 position of the furan ring a substituent having the formula —(CH₂)ₙ—NH—R′ where

n is 0 or 1;

R′ is H, (C₁-C₈)alkyl, (C₃-C₇)cycloalkyl, (C₂-C₈)alkenyl, (C₂-C₈)alkynyl, benzyl, aryl, (C₀-C₄)alkyl)-(C₁-C₆)heterocycloalkyl, (C₀-C₄)alkyl)-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl; and

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group.

The primary amine of Formula (III) can be, for instance, a 4-alkyl-3-aminopiperidine-2,6-dione such as 4-methyl-3-aminopiperidine-2,6-dione, 3-aminopiperidine-2,6-dione and salts thereof.

In other embodiments, the invention provides to a process as described in Scheme A described below for the preparation of a compound of Formula (I) or a pharmaceutically acceptable salt, solvate including a hydrate or polymorph thereof.

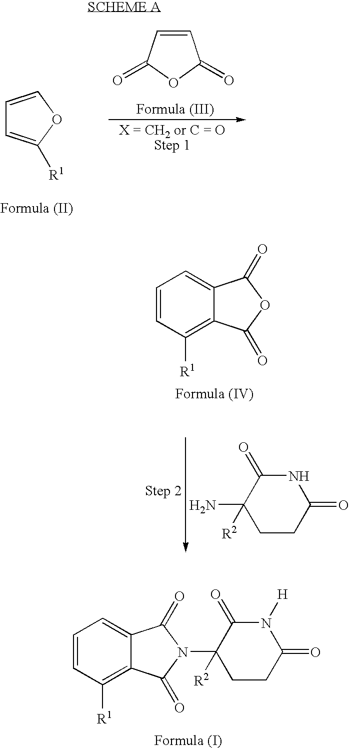

As depicted in Scheme A, the compound of Formula (I): 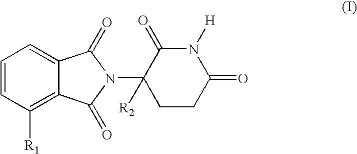
or a pharmaceutically acceptable salt, solvate including a hydrate or polymorph thereof, can be prepared by a process comprising the steps of:

(1) reacting a furan of Formula (II): 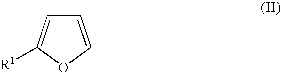
with maleic anhydride to form a compound of Formula (IV):

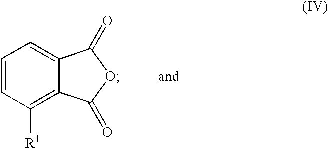

(2) reacting the compound of Formula (IV) with a primary amine having the formula: 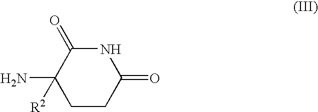
or a salt thereof, wherein:

R¹ is —(CH₂)ₙ—NH—R′;

R² is H, F, benzyl, (C₁-C₈)alkyl, (C₂-C₈)alkenyl, or (C₂-C₈)alkynyl;

R′ is H, (C₁₋C₈)alkyl, (C₃-C₇)cycloalkyl, (C₂-C₈)alkenyl, (C₂-C₈)alkynyl, benzyl, aryl, (C₀₋C₄)alkyl)-(C₁-C₆)heterocycloalkyl, (C₀-C₄)alkyl)-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl)-(C₁-C₆)heterocycloalkyl, (C₀-C₄)alkyl)-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group; and

n is 0 or 1.

In step 1 of Scheme A, the reaction between Formula (II) and maleic anhydride can occur in a solvent such as ethyl acetate, acetone, methyl ethyl ketone, diethyl ether, tetrahydrofuran, acetonitrile, dichloromethane, chloroform, N-methyl pyrrolidinone, dimethyl formamide, dimethyl sulfoxide and combinations thereof. In one embodiment, the solvent is ethyl acetate.

The reaction temperature can be between 20° C. and 80° C. In some embodiments of interest, the reaction temperature is between 30° C. and 70° C. In other embodiments of interest, the reaction temperature is between 40° C. and 60° C. In further embodiments of interest, the reaction temperature is between 45° C. and 55° C.

The reaction between Formula (II) and maleic anhydride can take place in the presence of an acid catalyst, such as trifluoroacetic acid, 4-(trifluoromethyl)benzoic acid, p-toluenesulfonic acid, methanesulfonic acid, acetic anhydride, and Lewis acids (e.g., Et₂AlCl, EtAlCl₂, BF₃, SnCl₄, AlCl₃, Ti (isopropoxide)₄ and TiCl₄). In one embodiment, the catalyst is trifluoroacetic acid.

The reaction time can vary from 1 to 24 hours, depending on the reaction temperature. In general, the higher the reaction temperature, the shorter is the reaction time. In one embodiment of interest, the reaction time is 8 hours at a reaction temperature between 18° C. and 24° C. In another embodiment of interest, the reaction time is 6 hours at a reaction temperature between 45° C. and 55° C.

In a preferred embodiment, the reaction between Formula (II) and maleic anhydride occurs in ethyl acetate at a temperature between 45° C. and 55° C. in the presence of trifluoroacetic acid for 6 hours. In another preferred embodiment, the reaction between Formula (II) and maleic anhydride occurs in ethyl acetate at room temperature in the presence of trifluoroacetic acid for 8 hours.

In general, any unsubstituted or 2-substituted furan compound that can undergo a Diels-Alder reaction with an alkene can be used for the reaction between Formula (II) and maleic anhydride. R¹ of Formula (II) can be —(CH₂)ₙ—NH—R′ wherein:

R′ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl(C₁-C₆)heterocycloalkyl, (C₀-C₄)alkyl)-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₀-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group; and

n is 0 or 1.

Non-limiting examples of the furan of Formula (II) include N-(2-furylmethyl)cyclopropanecarboxamide, N-(2-furylmethyl)cyclobutanecarboxamide, N-(2-furylmethyl)cyclopentanecarboxamide, N-(2-furylmethyl)cyclohexanecarboxamide, N-(2-furylmethyl)cycloheptanecarboxamide, 2-furaldehyde dimethylhydrazone, 2-furylmethanamine, N-isopentyl-2-furamide, N-(2-furylmethyl)-2,2-dimethylpropanamide, N-phenyl-2-furamide, N-(3-aminophenyl)-2-furamide, N-benzyl-2-furamide, N-(2-furylmethyl)benzamide, ethyl (2-furoylamino)acetate, N-(3-chlorophenyl)-2-furamide, N-cyclohexyl-N′-(2-furylmethyl)urea, 2-furyl methyl ether, 2-methylfuran, 2-aminofuran, 2-furonitrile, 2-furylmethanol, 2-furylacetonitrile, 2-nitrofuran, tert-butyl N-(2-furyl)carbamate, tert-butyl (furan-2-yl)methylcarbamate, 1-cyclohexyl-3-((furan-2-yl)methyl)thiourea, N-((furan-2-yl)methyl)picolinamide, N-((furan-2-yl)methyl)nicotinamide, 3-((furan-2-yl)methyl)-1,1-dimethylurea, 3-((furan-2-yl)methyl)-1,1-diethylurea, 1-((furan-2-yl)methyl)-1,3,3-trimethylurea, N-((furan-2-yl)methyl)piperidine-1-carboxamide, 1-((furan-2-yl)methyl)-3-(3-methoxyphenyl)-1-methylurea, 1-(3,4-dichlorophenyl)-3-((furan-2-yl)methyl)urea, 1-(3-chloro-4-methylphenyl)-3-((furan-2-yl)methyl)urea, 1-((furan-2-yl)methyl)-3-(naphthalen-2-yl)urea, N-((benzofuran-2-yl)methyl)furan-2-amine, N-((4,5-dimethylfuran-2-yl)methyl)furan-2-amine, 3-amino-N-((furan-2-yl)methyl)propanamide, N-((furan-2-yl)methyl)benzamide, N-(3,4-dimethoxyphenyl)furan-2-amine and 1-ethyl-3-((furan-2-yl)methyl)urea, all of which can be obtained commercially from a supplier, such as Aldrich Chemicals and Acros Organics, or be prepared by known synthetic methods using known starting materials. Preferred embodiments include N-(2-furylmethyl)cyclopropanecarboxamide, 2-furaldehyde dimethylhydrazone, 2-methylfuran and 2-furylmethanamine.

In some embodiments of interest, the furan of Formula (II) is selected from the group consisting of N-(2-furylmethyl)cyclohexanecarboxamide (Aldrich product # S904937), 2-methylfuran (Aldrich product # M46845) and 2-furylmethanamine (Aldrich product # F20009). In other embodiments of interest, the furan of Formula (II) is N-(2-furylmethyl)cyclopropanecarboxamide, N-(2-furylmethyl)cyclobutanecarboxamide, N-(2-furylmethyl)cyclopentanecarboxamide, N-(2-furylmethyl)cyclohexanecarboxamide, N-(2-furylmethyl)cyclopentylmethanecarboxamide, N-(2-furylmethyl)-1-methyl-cyclohexanecarboxamide, N-(2-furylmethyl)-2-cyclopentylethanecarboxamide, or N-(2-furylmethyl)cycloheptanecarboxamide, all of which can be prepared by reacting 2-furylmethanamine respectively with cyclopropanecarbonyl chloride, cyclobutanecarbonyl chloride, cyclopentanecarbonyl chloride, cyclohexanecarbonyl chloride, cyclopentylacetyl chloride, 1-methylcyclohexanecarbonyl chloride, 3-cyclopentylpropanoyl chloride, or cycloheptanecarbonyl chloride. All of the above-mentioned chlorides can be obtained commercially from a supplier such as Aldrich Chemicals.

In further embodiments, R¹ of the furan of Formula (II) is selected from the group consisting of H, alkyl, —C(R⁷)═N—NR⁸R⁹, —CHR⁷—NHR¹⁰ or an acid salt thereof, —CHR⁷—NHC(═O)R¹¹, —NHR¹² or an acid salt thereof and —OR¹³ where each of R⁷, R⁸, R⁹, R¹⁰, R¹¹, R¹², and R¹³ is independently hydrogen, alkyl, heteroalkyl, aryl, heteroaryl, cycloalkyl or heterocycloalkyl. In additional embodiments, R¹ of the furan of Formula (II) is selected from the group consisting of —CH═N—N(CH₃)₂, —CH₂NH₂ or an acid salt thereof, and —CH₂—C(═O)—R¹¹ where R¹¹ is cyclopropyl, cyclobutyl, cyclopentyl, 3-cyclopentylpropyl, cyclohexyl, 1-methylcyclohexyl, cycloheptyl, cyclooctyl, cyclodecyl or cyclopentylmethyl.

In step 2 of Scheme A, the reaction between Formula (IV) and the primary amine of Formula (III) or a salt thereof can occur in a solvent, such as ethyl acetate, acetone, methyl ethyl ketone, diethyl ether, tetrahydrofuran, acetic acid, acetonitrile, N-methyl pyrrolidinone, dimethylformamide, dimethyl sulfoxide and mixtures thereof. In one embodiment, the solvent is acetonitrile.

The reaction temperature can be between 20° C. and 100° C. In some embodiments of interest, the reaction temperature is between 40° C. and 90° C. In other embodiments of interest, the reaction temperature is between 60° C. and 90° C. In further embodiments of interest, the reaction temperature is between 75° C. and 85° C.

The reaction between Formula (IV) and the primary amine of Formula (III) or a salt thereof can occur in the presence of a catalyst. The catalyst can be selected from the group consisting of carboxylic acids (e.g., acetic acid, formic acid, and butanoic acid), metal carboxylates (e.g., sodium acetate and potassium formate), inorganic bases (e.g., sodium bicarbonate, potassium carbonate and lithium hydroxide), organic amines (e.g., triethylamine, pyridine, DBU, N,N-diisopropylethylamine (DIPEA) and imidazole) and combinations thereof. In one embodiment of interest, the catalyst is 1,8-diazabicyclo[5.4.0]undec-7-ene (DBU). In another embodiment of interest, the catalyst is imidazole. In a further embodiment of interest, the catalyst is a mixture of acetic acid and imidazole.

The reaction time can vary from 1 to 24 hours, depending on the reaction temperature. In general, the higher the reaction temperature, the shorter is the reaction time. In one embodiment of interest, the reaction time is between 2 and 3 hours at a reaction temperature between 78° C. and 82° C.

In one preferred embodiment, the reaction between Formula (IV) and the primary amine of Formula (III) or a salt thereof occurs in acetonitrile at a temperature between 78° C. and 82° C. for 2 and 3 hours in the presence of acetic acid and imidazole. In another preferred embodiment, the reaction between Formula (IV) and the primary amine or a salt thereof occurs in acetonitrile at a temperature between 78° C. and 82° C. for 2 and 3 hours in the presence of a mixture of acetic acid and imidazole in a molar ratio of 1:1.

In general, any primary amine of Formula (III) that can react with Formula (IV) can be used for this invention. Non-limiting examples of the primary amine of Formula (III) include 3-aminopiperidine-2,6-dione (i.e., α-aminoglutarimide), 4-alkyl-3-aminopiperidine-2,6-dione such as 3-amino-4-methyl-piperidine-2,6-dione and salts thereof. All of the above primary amines can be obtained commercially from a supplier, such as Aldrich chemicals (Milwaukee, Wis.) and Evotec OAI, (Oxfordshire, UK), or can be prepared by known synthetic methods. The primary amine can be in the form of a free amine or an acid salt, such as hydrochloride salt.

In preferred embodiments, the primary amine is selected from the group consisting of 3-amino-4-methyl-piperidine-2,6-dione, 3-aminopiperidine-2,6-dione and salts thereof. In a further embodiment, the primary amine is a racemic mixture. In an additional embodiment, the primary amine is enantiomerically pure such as the (+)-enantiomer. In another embodiment, the primary amine is enantiomerically pure such as the (−)-enantiomer.

If a racemic compound of Formula (I) is desired, a racemic primary amine of Formula (III) can be used in step 2. Conversely, if an enantiomerically pure compound of Formula (I) is desired, an enantiomerically pure primary amine of Formula (III) can be used in step 2. Alternatively, if an enantiomerically pure compound of Formula (I) is desired, a racemic mixture of Formula (I) can be prepared and then the racemic mixture can be resolved into the enantiomers by conventional resolution techniques such as biological resolution and chemical resolution. In general, biological resolution uses a microbe which metabolizes one specific enantiomer leaving the other enantiomer alone. In chemical resolution, the racemic mixture is converted into two diastereoisomers that can be separated by conventional techniques such as fractional crystallization and chromatographies. Once separated, the diasteriosomeric forms can be converted separately back to the enantiomers.

Similarly, if a racemic substituted 2-(2,6-dioxopiperidin-3-yl)isoindole-1,3-dione is desired, a racemic 3-aminopiperidine-2,6-dione can be used respectively in step 2. Conversely, if an enantiomerically pure substituted 2-(2,6-dioxopiperidin-3-yl)isoindole-1,3-dione is desired, an enantiomerically pure 3-aminopiperidine-2,6-dione can be used in step 2. Under the reaction conditions as described herein, the stereochemistry of any chiral stereocenter in a primary amine, such as aminopiperidine-2,6-dione ring and 3-amino-4-methyl-piperidine-2,6-dione, can be retained. In one embodiment, the compound of Formula (I) is a racemic mixture. In another embodiment, the compound of Formula (I) is the (+)-enantiomer. In a further embodiment, the compound of Formula (I) is the (−)-enantiomer.

In a particular embodiment of the compound of Formula (I) in Scheme A, R² is hydrogen. In further embodiments, R¹ of the compound of Formula (I) is selected from the group consisting of H, alkyl, —C(R⁷)═N—NR⁸R⁹, —CHR⁷—NHR¹⁰ or an acid salt thereof, —CHR⁷—NHC(═O)R¹¹, —NHR¹² or an acid salt thereof and —OR¹³, where each of R⁷, R⁸, R⁹, R¹⁰, R¹¹, R¹², and R¹³ is independently hydrogen, alkyl, heteroalkyl, aryl, heteroaryl, cycloalkyl or heterocycloalkyl. In other embodiments, R¹ of the compound of Formula (I) is selected from the group consisting of —CH═N—N(CH₃)₂, —CH₂NH₂ or an acid salt thereof, and —CH₂—C(═O)—R¹¹ where R¹¹ is cyclopropyl, cyclobutyl, cyclopentyl, 3-cyclopentylpropyl, cyclohexyl, 1-methylcyclohexyl, cycloheptyl, cyclooctyl, cyclodecyl or cyclopentylmethyl. In an additional embodiment, R¹ of the compound of Formula (I) is —C(R⁷)═N—NR⁸R⁹ where each of R⁷, R⁸ and R⁹ is independently hydrogen, alkyl, heteroalkyl, aryl, heteroaryl, cycloalkyl or heterocycloalkyl. In a further embodiment, each of R¹ and R² of Formula (I) is hydrogen.

In further embodiments, the invention provides a process as described in Scheme B below for the preparation of a compound of Formula (I) or a pharmaceutically acceptable salt, solvate including a hydrate or polymorph thereof.

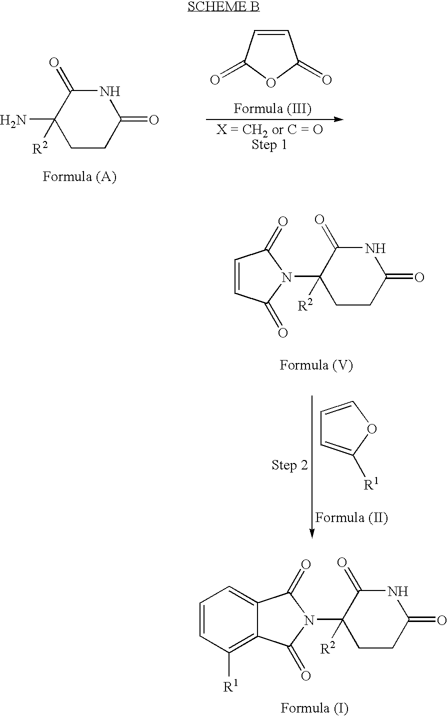

As depicted in Scheme B, the compound of Formula (I): 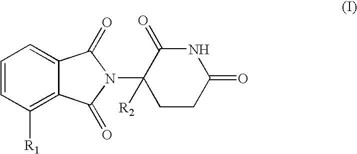
or a pharmaceutically acceptable salt, solvate including a hydrate or polymorph thereof, can be prepared by a process comprising the step of reacting a furan of Formula (II): 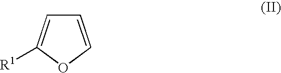
with a heterocyclic compound of Formula (V): 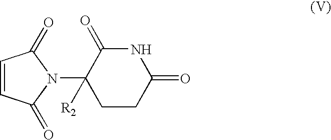
wherein R¹ and R² are the same as those described above in Scheme A.

The reaction between Formula (II) and Formula (V) can occur in a solvent, such as ethyl acetate, acetone, methyl ethyl ketone, diethyl ether, tetrahydrofuran, acetonitrile, N-methyl pyrrolidinone, dimethyl formamide, dimethyl sulfoxide and combinations thereof. In one embodiment, the solvent is ethyl acetate.

The reaction temperature can be between 20° C. and 80° C. In some embodiments of interest, the reaction temperature is between 30° C. and 70° C. In other embodiments of interest, the reaction temperature is between 40° C. and 60° C. In further embodiments of interest, the reaction temperature is between 45° C. and 55° C.

The reaction between Formula (II) and maleic anhydride can take place in the presence of an acid catalyst, such as trifluoroacetic acid, 4-(trifluoromethyl)benzoic acid, p-toluenesulfonic acid, methanesulfonic acid, acetic anhydride and Lewis acids (e.g., Et₂AlCl, EtAlCl₂, BF₃, SnCl₄, AlCl₃, Ti(isopropoxide)₄ and TiCl₄). In one embodiment, the catalyst is trifluoroacetic acid.

The reaction time can vary from 1 to 24 hours, depending on the reaction temperature. In general, the higher the reaction temperature, the shorter is the reaction time. In one embodiment of interest, the reaction time is 8 hours at a reaction temperature between 18° C. and 24° C. In another embodiment of interest, the reaction time is 6 hours at a reaction temperature between 45° C. and 55° C.

In a preferred embodiment, the reaction between Formula (II) and maleic anhydride occurs in ethyl acetate at a temperature between 45° C. and 55° C. in the presence of trifluoroacetic acid for 6 hours. In another preferred embodiment, the reaction between Formula (II) and maleic anhydride occurs in ethyl acetate at room temperature in the presence of trifluoroacetic acid for 8 hours.

In general, any unsubstituted or 2-substituted furan compound that can undergo a Diels-Alder reaction with an alkene can be used for this invention. The furan compound used in Scheme B can be the same as the furan compound of Formula (II) for Scheme A as described above. Preferred furan compounds of Formula (II) include N-(2-furylmethyl)cyclopropanecarboxamide, 2-furaldehyde dimethylhydrazone, 2-methylfuran and 2-furylmethanamine.

In some embodiments of interest, the furan of Formula (II) is selected from the group consisting of furan (Aldrich product # 185922), N-(2-furylmethyl)cyclohexanecarboxamide (Aldrich product # S904937), 2-methylfuran (Aldrich product # M46845) and 2-furylmethanamine (Aldrich product # F20009). In other embodiments of interest, the furan of Formula (II) is selected from the group consisting of N-(2-furylmethyl)cyclopropanecarboxamide, N-(2-furylmethyl)cyclobutanecarboxamide, N-(2-furylmethyl)cyclopentanecarboxamide, N-(2-furylmethyl)cyclohexanecarboxamide, N-(2-furylmethyl)cyclopentylmethanecarboxamide, N-(2-furylmethyl)-1-methyl-cyclohexanecarboxamide, N-(2-furylmethyl)-2-cyclopentylethanecarboxamide, and N-(2-furylmethyl)cycloheptanecarboxamide, all of which can be prepared by reacting 2-furylmethanamine respectively with cyclopropanecarbonyl chloride, cyclobutanecarbonyl chloride, cyclopentanecarbonyl chloride, cyclohexanecarbonyl chloride, cyclopentylacetyl chloride, 1-methylcyclohexanecarbonyl chloride, 3-cyclopentylpropanoyl chloride, and cycloheptanecarbonyl chloride. All of the above-mentioned chlorides can be obtained commercially from a supplier such as Aldrich Chemicals.

In other embodiments of interest, the heterocyclic compound of Formula (V) can be prepared according to any method known to those of skill in the art, such as step 1 of Scheme B. Based on the disclosure herein, a person skill in the art can used other known methods for the preparation of the heterocyclic compound of Formula (V). According to step 1 of Scheme B, the heterocyclic compound of Formula (V) can be prepared by the reaction of maleic anhydride with a primary amine having the formula: 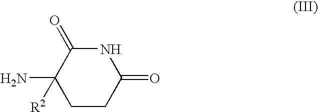
or a salt thereof, where R² is H, F, benzyl, (C₁-C₈)alkyl, (C₂-C₈)alkenyl, or (C₂-C₈)alkynyl.

The reaction between maleic anhydride and the primary amine of Formula (III) or a salt thereof can occur in a solvent, such as ethyl acetate, acetone, methyl ethyl ketone, diethyl ether, tetrahydrofuran, acetonitrile, N-methyl pyrrolidinone, dimethyl formamide, dimethyl sulfoxide and mixture thereof. In one embodiment, the solvent is acetonitrile.

The reaction temperature of the reaction between maleic anhydride and the primary amine of Formula (III) can be between 20° C. and 100° C. In some embodiments of interest, the reaction temperature is between 40° C. and 90° C. In other embodiments of interest, the reaction temperature is between 60° C. and 90° C. In further embodiments of interest, the reaction temperature is between 75° C. and 85° C.

The reaction between maleic anhydride and the primary amine of Formula (III) or a salt thereof can occur in the presence of a catalyst. The catalyst can be selected from the group consisting of carboxylic acids (e.g., acetic acid, formic acid, and butanoic acid), metal carboxylates (e.g., sodium acetate and potassium formate), inorganic bases (e.g., sodium bicarbonate, potassium carbonate and lithium hydroxide), organic amines (e.g., triethylamine, pyridine, DBU, DIPEA and imidazole) and combinations thereof. In one embodiment of interest, the catalyst is 1,8-diazabicyclo[5.4.0]undec-7-ene (DBU). In another embodiment of interest, the catalyst is imidazole. In a further embodiment of interest, the catalyst is a mixture of acetic acid and imidazole.

The reaction time of the reaction between maleic anhydride and the primary amine of Formula (III) can vary from 1 to 24 hours, depending on the reaction temperature. In general, the higher the reaction temperature, the shorter is the reaction time. In one embodiment of interest, the reaction time is between 2 and 3 hours at a reaction temperature between 78° C. and 82° C.

In one preferred embodiment, the reaction between maleic anhydride and the primary amine of Formula (III) or a salt thereof occurs in acetonitrile at a temperature between 78° C. and 82° C. for 2 and 3 hours in the presence of acetic acid and imidazole. In another preferred embodiment, the reaction between maleic anhydride and the primary amine of Formula (III) or a salt thereof occurs in acetonitrile at a temperature between 78° C. and 82° C. for 2 and 3 hours in the presence of a mixture of acetic acid and imidazole in a molar ratio of 1:1.

In general, any primary amine of Formula (III) that can react with maleic anhydride can be used for this invention. The primary amine used in Scheme B can be the same as the primary amine used in Scheme A as described above.

In some embodiments of interest, the primary amine of Formula (III) is selected from the group consisting of 3-aminopiperidine-2,6-dione, 3-amino-4-methyl-piperidine-2,6-dione and salts thereof. In a further embodiment, the primary amine is a racemic mixture. In an additional embodiment, the primary amine is enantiomerically pure such as the (+)-enantiomer. In another embodiment, the above primary amine is enantiomerically pure such as the (−)-enantiomer.

If a racemic compound of Formula (V) is desired, a racemic primary amine of Formula (III) can be used in step 2. Conversely, if an enantiomerically pure compound of Formula (V) is desired, an enantiomerically pure primary amine of Formula (III) can be used in step 2. Alternatively, if an enantiomerically pure compound of Formula (V) is desired, a racemic mixture of Formula (V) can be prepared and then the racemic mixture can be resolved into the enantiomers by conventional resolution techniques.

Similarly, if a racemic substituted 2-(2,6-dioxopiperidin-3-yl)isoindole-1,3-dione is desired, a racemic 3-aminopiperidine-2,6-dione can be used respectively in step 2. Conversely, if an enantiomerically pure substituted 2-(2,6-dioxopiperidin-3-yl)isoindole-1,3-dione is desired, an enantiomerically pure 3-aminopiperidine-2,6-dione can be used in step 2. Under the reaction conditions as described herein, the stereochemistry of any chiral stereocenter in a primary amine, such as the 3 position of the 3-aminopiperidine-2,6-dione, can be retained. In one embodiment, the compound of Formula (V) is a racemic mixture. In another embodiment, the compound of Formula (V) is enantiomerically pure such as the (+)-enantiomer. In a further embodiment, the compound of Formula (V) is enantiomerically pure such as the (−)-enantiomer.

In a particular embodiment of the compound of Formula (I) in Scheme B, R² is hydrogen. In further embodiments, R¹ of the compound of Formula (I) is selected from the group consisting of H, alkyl, —C(R⁷)═N—NR⁸R⁹, —CHR⁷—NHR¹⁰ or an acid salt thereof, —CHR⁷—NHC(═O)R¹¹, —NHR¹² or an acid salt thereof and —OR¹³, where each of R⁷, R⁸, R⁹, R¹⁰, R¹¹, R¹² and R¹³ is independently hydrogen, alkyl, heteroalkyl, aryl, heteroaryl, cycloalkyl or heterocycloalkyl. In other embodiments, R¹ of the compound of Formula (I) is selected from the group consisting of —CH═N—N(CH₃)₂, —CH₂NH₂ or an acid salt thereof, and —CH₂—C(═O)—R¹¹ where R¹¹ is cyclopropyl, cyclobutyl, cyclopentyl, 3-cyclopentylpropyl, cyclohexyl, 1-methylcyclohexyl, cycloheptyl, cyclooctyl, cyclodecyl or cyclopentylmethyl. In an additional embodiment, R¹ of the compound of Formula (I) is —C(R⁷)═N—NR⁸R⁹ where each of R⁷, R⁸ and R⁹ is independently hydrogen, alkyl, heteroalkyl, aryl, heteroaryl, cycloalkyl or heterocycloalkyl. In a further embodiment, each of R¹ and R² of Formula (I) is hydrogen.

In some embodiments, when R¹ of Formula (I) in Scheme A or B is —C(R⁷)═N—NR⁸R⁹ where each of R⁷, R⁸ and R⁹ is independently hydrogen, alkyl, heteroalkyl, aryl, heteroaryl, cycloalkyl or heterocycloalkyl, Scheme A or B can further comprise a reduction step that converts the —C(R⁷)═N—NR⁸R⁹ group into a —CHR⁷—NH₂ group. The reduction step can be represented by Scheme C below.

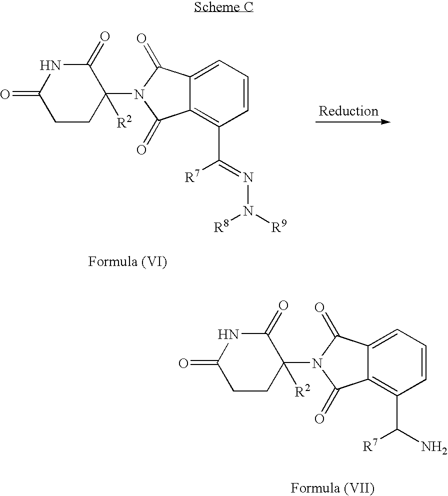

In Scheme C, Formula (VI) can be reduced by a reducing agent to form a compound of Formula (VII):

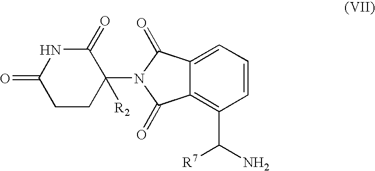

where R² is the same as those described above in Scheme A or B; and

each of R⁷, R⁸ and R⁹ is hydrogen, alkyl, heteroalkyl, aryl, heteroaryl, cycloalkyl or heterocycloalkyl.

The reduction of the —C(R⁷)═N—NR⁸R⁹ group of Formula (VI) to —CH(R⁷)—NH₂ can be effected under hydrogen with a catalyst. In one embodiment, the catalyst is a Pd catalyst. In another embodiment, the catalyst is 5% Pd/C. In a further embodiment, the catalyst is 10% Pd/C. Any other reducing agent known in the art for reducing a hydrazone to an amine can also be used for this reducing step.

In additional embodiment, the reduction occurs in the presence of an acid source such as methanesulfonic acid, trifluoroacetic acid, 4-(trifluoromethyl)benzoic acid, p-toluenesulfonic acid, hydrochloric acid, nitric acid, sulfuric acid and phosphoric acid. In a particular embodiment, the acid source is methanesulfonic acid.

The reduction can occur in a solvent. In one embodiment, the reduction is conducted in a protic solvent, such as alcohols, water, and combinations thereof. In a further embodiment, the alcohol solvent is selected from the group consisting of methanol, ethanol, propanol, isopropanol, butanol, isobutanol, t-butanol and combinations thereof. In an additional embodiment, the solvent is a mixture of an alcohol and water. In one embodiment, the solvent is a mixture of methanol and water in a volume ratio between 1:5 and 5:1. In a particular embodiment, the solvent is a mixture of methanol and water in a volume ratio of 2:1. In another embodiment, the reduction is conducted in an apolar, aprotic solvent. The solvent is 1,4-dioxane in a particular embodiment. In yet another embodiment, the reduction is conducted in a polar, aprotic solvent. The solvent is acetone in a particular embodiment. In another embodiment, the solvent is DMSO, DMF or THF.

The reduction is generally carried out at a hydrogen pressure that drives the reaction to substantial completion. In a particular embodiment, the reduction is carried out at a hydrogen pressure between about 2.7 and 3.5 bars (about 40 and 50 psi or about 5332 and 6666 pascals).

In one embodiment, the reduction is run at ambient temperature. The reduction is generally performed until the reaction is substantially complete. In a particular embodiment, the reduction is performed for at least about 16-18 hours at a temperature between 18° C. to 24° C.

In a preferred embodiment, the reduction occurs at a temperature between 18° C. to 24° C. for 16-18 hours in a mixture of methanol and water in a volume ratio of 2:1 and in the presence of 10% Pd/C and methanesulfonic acid. In a further preferred embodiment, the reduction occurs at a pressure between about 40 and 50 psi or 2.7 to 3.5 bars.

Optionally, the compound of Formula (VII) can be converted into an acid salt by reacting the compound of Formula (VII) with an acid in a molar ratio of 1:1. Non-limiting examples of suitable acid include methanesulfonic acid, trifluoroacetic acid, 4-(trifluoromethyl)benzoic acid, p-toluenesulfonic acid, hydrochloric acid, nitric acid, sulfuric acid and phosphoric acid. In one embodiment, the compound of Formula (VII) is converted into a hydrochloride salt with 12N hydrochloric acid at a temperature between 0° C. and 22° C.

In some embodiments of interest, the compound of Formula (VII) or its acid salt can be acylated with an acylating agent to form an acylated compound of Formula (VIII). Scheme D below illustrates one possible way to convert the —CH(R⁷)—NH₂ group or its salt into —CHR⁷—NHC(═O)R¹¹ with an acyl halide where each of R⁷ and R¹¹ is independently hydrogen, alkyl, heteroalkyl, aryl, heteroaryl, cycloalkyl, heterocycloalkyl or a combination thereof.

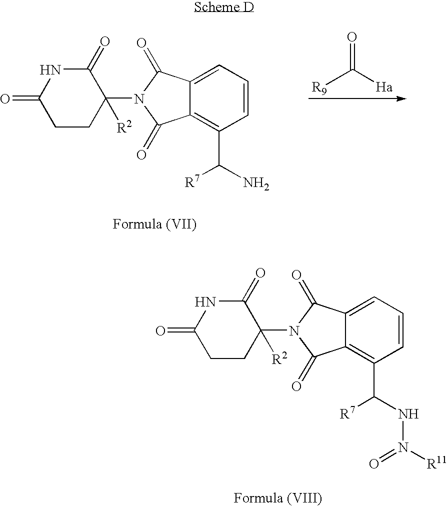

In one embodiment of Scheme D, the —CH(R⁷)—NH₂ group of Formula (VII) reacts with an acyl halide having the formula R¹¹—C(═O)-Ha to form the —CH(R⁷)—NHC(═O)—R¹¹ group of Formula (VIII) where Ha is F, Cl, Br or I; and R¹¹ is independently hydrogen, alkyl, heteroalkyl, aryl, heteroaryl, cycloalkyl, heterocycloalkyl or a combination thereof. In another embodiment of Scheme D, the —CH(R⁷)—NH₂ group of Formula (VII) is in an acid salt form, such as a hydrochloric acid salt, and reacts with an acyl halide to form the —CH(R⁷)—NHC(═O)—R¹¹ group.

The reaction between the compound of Formula (VII) or its acid salt and the acyl halide can occur in a solvent, such as ethyl acetate, acetone, methyl ethyl ketone, diethyl ether, tetrahydrofuran, acetonitrile, dichloromethane, chloroform, N-methyl pyrrolidinone, dimethyl formamide, dimethyl sulfoxide and mixture thereof. In one embodiment, the solvent is acetonitrile.

The reaction temperature of the reaction between the acyl halide and the compound of Formula (VII) or its acid salt can be between 0° C. and 40° C. In one embodiment of interest, the reaction temperature is between 0° C. and 24° C.

The reaction between the compound of Formula (VII) or its acid salt and the acyl halide can occur in the presence of a base catalyst, such as organic amines. Non-limiting examples of organic amines include N,N-diisopropylethylamine, triethylamine, pyridine and DBU, imidazole, and combinations thereof. In one embodiment of interest, the catalyst is triethylamine. In another embodiment of interest, the catalyst is imidazole. In a further embodiment of interest, the catalyst is N,N-diisopropylethylamine.

The reaction time of the reaction between the compound of Formula (VII) or its acid salt and the acyl halide can vary from 1 to 24 hours, depending on the reaction temperature. In general, the higher the reaction temperature, the shorter is the reaction time. In one embodiment of interest, the reaction time is between 3 and 4 hours at a reaction temperature between 0° C. and 24° C.

In one embodiment, the acyl chloride is added to a solution of the compound of Formula (VII), followed by the addition of the base catalyst. In another embodiment, the base catalyst is added to a solution of the compound of Formula (VII), followed by the addition of the acyl chloride. In another embodiment, the molar ratio of the base catalyst to the compound of Formula (VII) is 1:1. In an additional embodiment, the molar ratio of the base catalyst to the hydrochloric acid salt of the compound of Formula (VII) is 2:1.

In general, any acyl halide that can react with a primary amine or a secondary amine can be used for this embodiment. Non-limiting examples of the acyl halide include cyclopropanecarbonyl chloride, cyclobutanecarbonyl chloride, cyclopentanecarbonyl chloride, cyclohexanecarbonyl chloride, cyclopentylacetyl chloride, 1-methylcyclohexanecarbonyl chloride, 3-cyclopentylpropanoyl chloride, and cycloheptanecarbonyl chloride, all of which can be obtained commercially from a supplier, such as Aldrich Chemicals, Milwaukee, Wis. or be prepared by halogenating the corresponding carboxylic acids (R¹¹COOH) with a halogenating agent. The halogenating agent can be PY₃, PY₅ or SOY₂ where Y can be F, Cl, Br or I. For example, an acyl chloride (such as cycloheptanecarbonyl chloride) can be prepared by reacting the corresponding carboxylic acid (cycloheptanecarboxylic acid) with SOCl₂ or PCl₅. Similarly, an acyl bromide can be prepared by reacting the corresponding carboxylic acid with PBr₅.

The acylated compound of Formula (VIII) can be purified by recrystallization with a solvent. In one embodiment, the solvent is N-methyl pyrrolidinone, methanol, ethyl acetate, isopropanol, ethanol, acetic acid, water or a combination thereof. In a further embodiment, the solvent is a mixture of N-methyl pyrrolidinone and methanol in a volume ratio of 3:1 to 1:3. In a further embodiment, the solvent is a mixture of N-methyl pyrrolidinone and ethyl acetate in a volume ratio of 3:1 to 1:3. In a further embodiment, the solvent is a mixture of N-methyl pyrrolidinone and ethanol in a volume ratio of 3:1 to 1:3. In a further embodiment, the solvent is a mixture of N-methyl pyrrolidinone and isopropanol in a volume ratio of 3:1 to 1:3. In a further embodiment, the solvent is a mixture of acetic acid and ethanol in a volume ratio of 2:1 to 1:2. In a further embodiment, the solvent is a mixture of acetic acid and water in a volume ratio of 2:1 to 1:2. In a further embodiment, the solvent is acetic acid. In a preferred embodiment, the solvent is a mixture of N-methyl pyrrolidinone and water in a volume ratio of 2:1 to 1:2 by weight, more preferably in a volume ratio of 1:1.5 to 1.5:1.

Particular embodiments of the present invention are illustrated by the syntheses of the therapeutically 4-[(N,N-dimethylhydrazono)methyl]-2-(2,6-dioxopiperidin-3-yl)isoindole-1,3-dione, 4-aminomethyl-2-(2,6-dioxopiperidin-3-yl)isoindole-1,3-dione or an acid salt thereof, and 4-(cyclopropanecarbonylamino)methyl-2-(2,6-dioxopiperidin-3-yl)isoindole-1,3-diones as shown in Scheme E below. Modifications of variables including, but not limited to, reaction solvents, reaction times, reaction temperatures, reagents, starting materials, and functional groups in the particular embodiments of the synthesis of 4-[(N,N-dimethylhydrazono)methyl]-2-(2,6-dioxopiperidin-3-yl)isoindole-1,3-dione, 4-aminomethyl-2-(2,6-dioxopiperidin-3-yl)isoindole-1,3-dione or an acid salt thereof, and 4-(cyclopropanecarbonylamino)methyl-2-(2,6-dioxopiperidin-3-yl)isoindole-1,3-diones will be apparent to those of ordinary skill in the art.

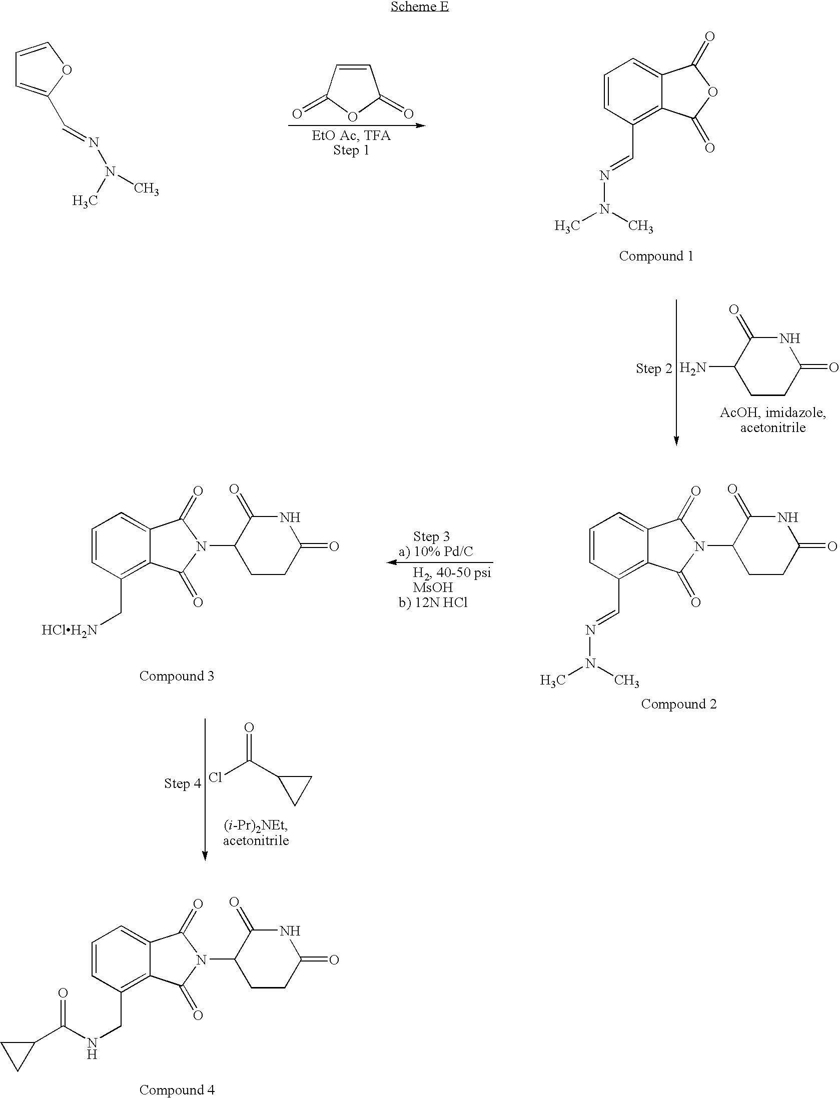

In the first step of Scheme E, maleic anhydride reacts with 2-furaldehyde dimethylhydrazone in the presence of trifluoroacetic acid in ethyl acetate at a temperature between 45° C. and 55° C. to form 4-[(N,N-dimethylhydrazono)methyl]isobenzofuran-1,3-dione (Compound 1).

In the second step of Scheme E, Compound 1 reacts with 3-aminopiperidine-2,6-dione hydrochloride (i.e., α-amino glutarimide hydrochloride) in the presence of a mixture of acetic acid and imidazole in acetonitrile at a temperature between 75° C. and 85° C. to form 4-[(N,N-dimethylhydrazono)methyl]-2-(2,6-dioxopiperidin-3-yl)isoindole-1,3-dione (Compound 2).

In the third step of Scheme E, the —CH═N—N(CH₃)₂ group of Compound 2 is reduced to a —CH₂NH₂ group by hydrogen in the presence of 10% Pd/C to form 4-aminomethyl-2-(2,6-dioxopiperidin-3-yl)isoindole-1,3-dione. The reduction reaction is carried out in the presence of methanesulfonic acid so that the 4-aminomethyl-2-(2,6-dioxopiperidin-3-yl)isoindole-1,3-dione is in the form of a mesylate salt. Next, the mesylate salt is converted to the corresponding hydrochloride salt (Compound 3) by 12N hydrochloric acid at a temperature between 0° C. and 24° C. The reduction reaction can occur in a mixture of methanol and water. The pressure of hydrogen can be between about 40 and 50 psi (about 2.7 and 3.5 bars).

In the fourth step of Scheme E, Compound 3 is acylated with cyclopropanecarbonyl chloride in the presence of N,N-diisopropylethylamine in acetonitrile at a temperature between 0° C. and 20° C. so as to form 4-[(cyclopropanecarbonylamino)methyl]-2-(2,6-dioxopiperidin-3-yl)isoindole-1,3-diones (Compound 4).

In some embodiments of interest, R¹ in Formulae (I), (II), or (IV) comprises —(CH₂)ₙ—NH—R′ wherein:

R′ is H, (C₁-C₈)alkyl, (C₃-C₇)cycloalkyl, (C₂-C₈)alkenyl, (C₂-C₈)alkynyl, benzyl, aryl, (C₀-C₄)alkyl-C₁-C₆)heterocycloalkyl, (C₀-C₄)alkyl)-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl)-(C₁-C₆)heterocycloalkyl, (C₀-C₄)alkyl)-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl)-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group; and

n is 0 or 1. In further embodiments of interest, R¹ in Formulae (I), (II), or (IV) is H.

### 5. EXAMPLES

### Synthesis of Substituted isoindole-1,3-diones

### Example 1

### Preparation of 4-[(N,N-dimethylhydrazono)methyl] isobenzofuran-1,3-dione (Compound 1)

Maleic anhydride (2) (277.5 g, 2.83 moles, from Aldrich Chemicals, Milwaukee, Wis.) and ethyl acetate (1050 ml) were charged into a 5 L three-necked flask at room temperature under nitrogen. A solution of 2-furaldehyde N,N-dimethylhydrazone (300 g, 2.2 moles, from Aldrich Chemicals, Milwaukee, Wis.) in ethyl acetate (450 ml) was charged into the flask. After the reaction mixture was stirred for 5-10 minutes, trifluoroacetic acid (12.4 g, 0.11 mole, 5 mol %, from Aldrich Chemicals, Milwaukee, Wis.) was charged into the flask over 15-20 minutes. A latent exotherm (˜15-25° C. above room temperature) was observed. After the exotherm had subsided, the reaction mixture was heated to 45-55° C. for 6 hours, or alternatively, the reaction mixture was stirred for 8 hours at room temperature. At end of the respective reaction period (8 hours for room temperature reaction or 6 hours for the heated reaction), the reaction mixture was cooled to room temperature if necessary. After the reaction mixture was filtered at room temperature under vacuum, the filtered solid was washed sequentially with 600 ml of ethyl acetate, 2.4 L of deionized water, and 600 ml of heptane. The solid was dried in a tray at 55-60° C. under vacuum for 8-12 hours. The yield of Compound 1 was found to be 400 g (84%) based on 277.5 g input of maleic anhydride (HPLC indicated 99.2% purity by peak area).

### Example 2

### Preparation of 4-[(N,N-dimethylhydrazono)methyl]isobenzofuran-1,3-dione (Compound 1)

Alternatively, Compound 1 was prepared similarly according to the above procedure for Example 1 except that trifluoroacetic acid (5 mol %) was replaced with SnCl₄ (0.08 mol %, from Aldrich Chemicals, Milwaukee, Wis.) and the reaction temperature and time are room temperature and 16-18 hours respectively. The yield of Compound 1 was found to be 65-68% based on 277.5 g input of maleic anhydride (HPLC indicated 99.2% purity by peak area).

### Example 3

### Preparation of 4-[(N,N-dimethylhydrazono)methyl]isobenzofuran-1,3-dione (Compound 1)

Alternatively, Compound 1 was prepared similarly according to the above procedure for Example 1 except that trifluoroacetic acid (5 mol %) was replaced with methanesulfonic acid (1 mol %, from Aldrich Chemicals, Milwaukee, Wis.) and the reaction temperature and time are room temperature and 16-18 hours respectively. The yield of Compound 1 was found to be 88-90% based on 277.5 g input of maleic anhydride (HPLC indicated 99.2% purity by peak area).

### Example 4

### Preparation of 4-[(N,N-dimethylhydrazono)methyl]-2-(2,6-dioxopiperidin-3-yl)isoindole-1,3-dione (Compound 2)

Compound 1 (300 g, 1.38 moles, prepared previously) was charged into a 5 L three-necked flask, followed by the addition of α-amino glutarimide hydrochloride (189 g, 1.15 mol, from Evotec OAI, Oxfordshire, UK), imidazole (780 g, 11.5 mol, from Aldrich Chemicals, Milwaukee, Wis.) and acetonitrile (2.28 L, from Fisher Scientific, Pittsburgh, Pa.), at room temperature under nitrogen to form a solution. After acetic acid (688 g, 11.5 mol, from Fisher Scientific, Pittsburgh, Pa.) was charged into the solution at room temperature, the reaction mixture was stirred for 10-15 minutes. An exotherm (˜10-15° C. above room temperature) was observed. After the exotherm had subsided, the reaction mixture was heated to 75-82° C. for 2-3 hours while the H₂O formed during the reaction was removed by distilling out 378 ml of an acetonitrile/water azeotrope. Next, the reaction mixture was cooled to 65° C. and diluted with water (756 ml) while it was stirred at room temperature. The reaction mixture was filtered under vacuum and the filtered solid was washed sequentially with deionized water (1512 ml) and heptane (378 ml). The solid was dried in a tray at 55-60° C. under vacuum for 8-12 hours. The yield of Compound 2 was found to be 311 g (83%) based on 189 g input of α-amino glutarimide hydrochloride (HPLC indicated 99.5% purity by peak area).

### Example 5

### Preparation of 4-aminomethyl-2-(2,6-dioxopiperidin-3-yl)isoindole-1,3-dione hydrochloride salt (Compound 3)

Compound 2 (100 g, 0.304 mol, prepared previously) was charged into a 5 L Parr-vessel, followed by the addition of 10% Pd/C (50% wet, 4 g, 4 wt %, from Johnson Matthey, London, UK), a mixture of methanol and water in a volume ratio of 2:1 (1500 ml), and methanesulfonic acid (58.5 g, 0.609 mol, from Aldrich Chemicals, Milwaukee, Wis.) at room temperature under nitrogen. The reaction mixture was purged with sequentially with nitrogen (3 times) and hydrogen (3 times). The reaction mixture was stirred at room temperature over 18-20 hours with hydrogen maintained at a pressure between 40-50 psi. Alternatively, the reaction mixture was stirred at 40° C. over 6-8 hours with hydrogen maintained at a pressure between 40-50 psi. Next, the reaction was filtered through a celite bed (1 inch thickness) and the celite bed was washed with a mixture of methanol and water in a volume ratio of 2:1 (200 ml). The reaction mixture was cooled to room temperature if necessary and then filtered. The filtrate was concentrated under reduced pressure (15-20 torr) at 35-45° C. until 1.36 L (80%) of the methanol and water mixture was collected. After the concentrated filtrate was diluted with acetone (500 ml) and cool in an ice-bath at 0-5° C., 12N hydrochloric acid (102 ml, 1.22 mol) was added at a rate such that the reaction temperature was maintained between 0 and 5° C. Next, the acetone solution was warmed to room temperature. When turbidity was observed in the acetone solution, 2 g (2 wt. %) of Compound 3 was added. The mixture was stirred at room temperature for 15 hours while Compound 3 precipitated out from the acetone solution. The mixture was charged with ethyl acetate (300 ml) and stirred for a further 2 hours at room temperature. The mixture was filtered and washed sequentially with acetonitrile (100 ml), ethyl acetate (100 ml) and heptane (100 ml). The filtered solid was dried in a tray at 55-60° C. under vacuum for 12 hours. The yield of Compound 3 was found to be 77 g (78%) based on 100 g input of Compound 2 (HPLC indicated 98% purity by peak area).

### Example 6

### Preparation of 4-aminomethyl-2-(2,6-dioxopiperidin-3-yl)isoindole-1,3-dione hydrochloride salt (Compound 3)

Alternatively, Compound 3 was prepared similarly according to the above procedure for Example 5 except that the mixture of methanol and water in a volume ratio of 2:1 was replaced with a mixture of acetic acid and water in a volume ratio of 1.5:1. The yield of Compound 3 was found to be 89% based on 100 g input of compound 2 (HPLC indicated 98% purity by peak area).

### Example 7

### Preparation of 4-aminomethyl-2-(2,6-dioxopiperidin-3-yl)isoindole-1,3-dione hydrochloride salt (Compound 3)

Alternatively, Compound 3 was prepared similarly according to the above procedure for Example 5 except that methanesulfonic acid was replaced with hydrochloric acid. The yield of Compound 3 was found to be 68% based on 1100 g input of compound 2 (HPLC indicated 98% purity by peak area).

### Example 8

### Preparation of 4-[(cyclopropanecarbonylamino)methyl]-2-(2,6-dioxopiperidin-3-yl)isoindole-1,3-diones (Compound 4)

After Compound 3 (100.0 g, 0.31 moles, prepared previously) and acetonitrile (1.0 L) were charged into a 5 L three-necked flask, the reaction mixture was cooled to 0-5° C. Next, cyclopropanecarbonyl chloride (35.5 g, 30.8 ml, 0.34 mole, from Aldrich Chemicals, Milwaukee, Wis.) was added to the cooled reaction mixture over 20-30 minutes at 0-5° C. with stirring. N,N-diisopropylethylamine (79.9 g, 107.7 ml, 0.62 mole, from Aldrich Chemicals, Milwaukee, Wis.) was added to the reaction mixture over 45-60 minutes while the temperature was maintained at 0-5° C. The reaction mixture was warmed to 18-22° C. and stirred for 3 additional hours until the reaction was complete. After the reaction mixture was cooled to 0-5° C., 2N aqueous hydrochloric acid (1.0 L) was added over 20-30 minutes while the temperature was maintained at 0-5° C. The reaction mixture was stirred for 1 hour while the reaction mixture gradually increased to 18-22° C. A white solid precipitated and was filtered out under vacuum and washed with 1.0 L deionized water. The white solid was dried in a tray at 50-55° C. under a pressure of 100-125 mm of Hg. The yield of Compound 4 was found to be 100.95 g (92%) based on 100 g input of Compound 3 (HPLC indicated 98.94% purity by peak area).

### Example 9

### Preparation of 4-aminomethyl-2-(2,6-dioxopiperidin-3-yl)isoindole-1,3-dione hydrochloride salt (Compound 4)

Alternatively, Compound 4 was prepared similarly according to the above procedure for Example 8 except that acetonitrile was replaced with tetrahydrofuran. The yield of Compound 4 was found to be 87% based on 100 g input of Compound 3 (HPLC indicated 98.94% purity by peak area).

### Example 10

### Preparation of 4-aminomethyl-2-(2,6-dioxopiperidin-3-yl)isoindole-1,3-dione hydrochloride salt (Compound 4)

Alternatively, Compound 4 was prepared similarly according to the above procedure for Example 8 except that acetonitrile was replaced with N-methyl pyrrolidinone. The yield of Compound 4 was found to be 88% based on 100 g input of Compound 3 (HPLC indicated 98.94% purity by peak area).

This invention is not to be limited in scope by the specific embodiments disclosed in the examples that are intended as illustrations of a few aspects of the invention and any embodiments that are functionally equivalent are within the scope of this invention. Indeed, various modifications of the invention in addition to those described herein will become apparent to skilled artisans and are intended to fall within the appended claims.

What is claimed is:

1. A process for preparing a compound of Formula (I): 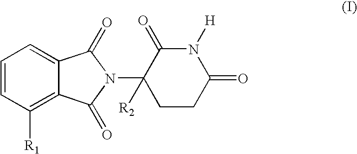
or a pharmaceutically acceptable salt, solvate including a hydrate or polymorph thereof, comprising the steps of:
(1) reacting a furan of Formula (II): 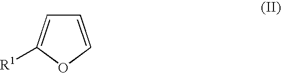
with maleic anhydride to form a compound of Formula (IV): 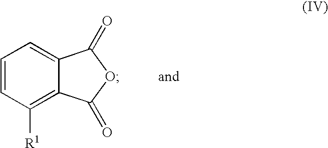
(2) reacting the compound of Formula (IV) with a primary amine having the formula: 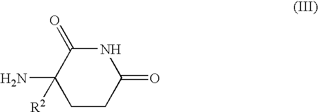
or a salt thereof, wherein:
R¹ is —(CH₂)ₙ—NH—R′;
R² is H, F, benzyl, (C₁-C₈)alkyl, (C₂-C₈)alkenyl, or (C₂-C₈)alkynyl;
R′ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR², C(S)NHR³, C(O)NR³R^{3′}, C(S)NR³R^{3′} or (C₁-C₈)alkyl-O(CO)R⁵;
R³ and R^{3′} are independently (C₁₋C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₂-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group; and
n is 0 or 1.

2. The process of claim 1, wherein the compound of Formula 1 is a racemic mixture, the (+)-enantiomer or the (−)-enantiomer.

3. The process of claim 1, wherein the primary amine or a salt thereof is a racemic mixture, the (+)-enantiomer or the (−)-enantiomer.

4. The process of claim 1, wherein R¹ is H, alkyl, —C(R⁷)═N—NR⁸R⁹, —CHR⁷—NHR¹⁰ or an acid salt thereof, —CHR⁷—NHC(═O)R¹¹, —NHR¹² or an acid salt thereof, or —OR¹³, where each of R⁷, R⁸, R⁹, R¹⁰, R¹¹, R¹² and R¹³ is independently hydrogen, alkyl, heteroalkyl, aryl, heteroaryl, cycloalkyl or heterocycloalkyl.

5. The process of claim 4, wherein R¹ is —CH═N—N(CH₃)₂, —CH₂NH₂ or an acid salt thereof, or —CH₂—C(═O)—R¹¹ where R¹¹ is cyclopropyl, cyclobutyl, cyclopentyl, 3-cyclopentylpropyl, cyclohexyl, 1-methylcyclohexyl, cycloheptyl, cyclooctyl, cyclodecyl or cyclopentylmethyl.

6. The process of claim 5, wherein R² is hydrogen.

7. The process of claim 1, wherein the furan of Formula (II) is N-(2-furylmethyl)cyclopropanecarboxamide, N-(2-furylmethyl)cyclobutanecarboxamide, N-(2-furylmethyl)cyclopentanecarboxamide, N-(2-furylmethyl)cyclohexanecarboxamide, N-(2-furylmethyl)cycloheptanecarboxamide, 2-furaldehyde dimethylhydrazone, 2-furylmethanamine, N-isopentyl-2-furamide, N-(2-furylmethyl)-2,2-dimethylpropanamide, N-phenyl-2-furamide, N-(3-aminophenyl)-2-furamide, N-benzyl-2-furamide, N-(2-furylmethyl)benzamide, ethyl (2-furoylamino)acetate, N-(3-chlorophenyl)-2-furamide, N-cyclohexyl-N′-(2-furylmethyl)urea, 2-furyl methyl ether, 2-methylfuran, 2-aminofuran, 2-furonitrile, 2-furylmethanol, 2-furylacetonitrile, 2-nitrofuran, tert-butyl N-(2-furyl)carbamate, tert-butyl (furan-2-yl)methylcarbamate, 1-cyclohexyl-3-((furan-2-yl)methyl)thiourea, N-((furan-2-yl)methyl)picolinamide, N-((furan-2-yl)methyl)nicotinamide, 3-((furan-2-yl)methyl)-1,1-dimethylurea, 3-((furan-2-yl)methyl)-1,1-diethylurea, 1-((furan-2-yl)methyl)-1,3,3-trimethylurea, N-((furan-2-yl)methyl)piperidine-1-carboxamide, 1-((furan-2-yl)methyl)-3-(3-methoxyphenyl)-1-methylurea, 1-(3,4-dichlorophenyl)-3-((furan-2-yl)methyl)urea, 1-(3-chloro-4-methylphenyl)-3-((furan-2-yl)methyl)urea, 1-((furan-2-yl)methyl)-3-(naphthalen-2-yl)urea, N-((benzofuran-2-yl)methyl)furan-2-amine, N-((4,5-dimethylfuran-2-yl)methyl)furan-2-amine, 3-amino-N-((furan-2-yl)methyl)propanamide, N-((furan-2-yl)methyl)benzamide, N-(3,4-dimethoxyphenyl)furan-2-amine or 1-ethyl-3-((furan-2-yl)methyl)urea.

8. The process of claim 1, wherein the reaction between Formula (II) and maleic anhydride occurs in a solvent selected from the group consisting of ethyl acetate, acetone, methyl ethyl ketone, diethyl ether, tetrahydrofuran, ethanol, methanol, acetonitrile, N-methyl pyrrolidinone, dimethyl formamide, dimethyl sulfoxide, and combinations thereof.

9. The process of claim 1, wherein the reaction between Formula (II) and maleic anhydride occurs at a temperature between 20° C. and 80° C.

10. The process of claim 1, wherein the reaction between Formula (II) and maleic anhydride occurs in the presence of an acid catalyst.

11. The process of claim 10, wherein the acid catalyst is selected from the group consisting of trifluoroacetic acid, 4-(trifluoromethyl)benzoic acid, p-toluenesulfonic acid, methanesulfonic acid, acetic anhydride, Lewis acids, and combinations thereof.

12. The process of claim 1, wherein the reaction between Formula (IV) and the primary amine or a salt thereof occurs at a temperature between 20° C. and 100° C.

13. The process of claim 1, wherein the reaction between Formula (IV) and the primary amine or a salt thereof occurs in the presence of a catalyst.

14. The process of claim 13, wherein the catalyst is selected from the group consisting of acetic acid, metal acetates, pyridine, sodium bicarbonate, triethylamine, 1,8-diazabicyclo[5.4.0]undec-7-ene, imidazole, and combinations thereof.

15. The process of claim 1, wherein the reaction between Formula (IV) and the primary amine or a salt thereof occurs in a solvent selected from the group consisting of ethyl acetate, acetone, methyl ethyl ketone, diethyl ether, tetrahydrofuran, ethanol, methanol, acetonitrile, N-methyl pyrrolidinone, dimethyl formamide, dimethyl sulfoxide, and combinations thereof.

16. The process of claim 1, wherein the primary amine is 3-aminopiperidine-2,6-dione, 3-amino-4-methyl-piperidine-2,6-dione or a salt thereof.

17. The process of claim 1, wherein R¹ is —C(R⁷)═N—NR⁸R⁹ where each of R⁷, R⁸ and R⁹ is independently hydrogen, alkyl, heteroalkyl, aryl, heteroaryl, cycloalkyl or heterocycloalkyl.

18. The process of claim 17 further comprising a third step of reducing the —C(R⁷)═N—NR⁸R⁹ group of Formula (I) to —CH(R⁷)—NH₂ by a reducing agent so as to form a compound of Formula (VII):

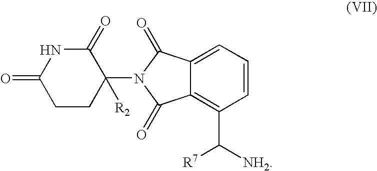

19. The process of claim 18, wherein each of R² and R⁷ is hydrogen.

20. The process of claim 18, wherein the reducing agent is hydrogen with a catalyst.

21. The process of claim 20, wherein the catalyst is a Pd catalyst.

22. The process of claim 21, wherein the Pd catalyst is 10% Pd/C.

23. The process of claim 18, wherein the third step occurs in the presence of methanesulfonic acid.

24. The process of claim 18, wherein the third reducing step occurs in a protic solvent, selected from the group consisting of alcohols, water, and combinations thereof.

25. The process of claim 18 further comprising a fourth step of acylating the —CH(R⁷)—NH₂ group of Formula (VII) with an acyl halide having the formula R⁷—C(═O)-Ha, where Ha is F, Cl, Br or I; and R¹¹ is hydrogen, alkyl, heteroalkyl, aryl, heteroaryl, cycloalkyl or heterocycloalkyl.

26. The process of claim 25 wherein the acyl halide is cyclopropanecarbonyl chloride, cyclobutanecarbonyl chloride, cyclopentanecarbonyl chloride, cyclohexanecarbonyl chloride, cyclopentylacetyl chloride, 1-methylcyclohexanecarbonyl chloride, 3-cyclopentylpropanoyl chloride or cycloheptanecarbonyl chloride.

27. The process of claim 25, wherein the fourth acylating step occurs in a solvent selected from the group consisting of ethyl acetate, acetone, methyl ethyl ketone, diethyl ether, tetrahydrofuran, ethanol, methanol, acetonitrile, N-methyl pyrrolidinone, dimethyl formamide, dimethyl sulfoxide, and combinations thereof.

28. The process of claim 25, wherein the fourth acylating step occurs at a temperature between 0° C. and 40° C.

29. The process of claim 25, wherein the fourth acylating step occurs in the presence of a base catalyst.

30. The process of claim 29, wherein the base catalyst is an organic amine.

31. A process for preparing a compound of Formula (I): 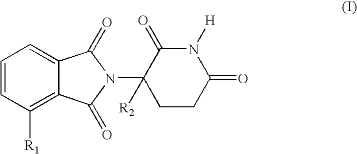
or a pharmaceutically acceptable salt, solvate including a hydrate or polymorph thereof, comprising the step of reacting a furan of Formula (II): 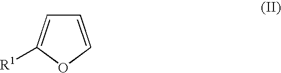
with a heterocyclic compound of Formula (V): 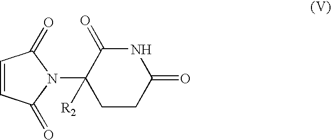
wherein:
R¹ is —(CH₂)ₙ—NH—R′;
R² is H, F, benzyl, (C₁-C₈)alkyl, (C₂-C₈)alkenyl, or (C₂-C₈)alkynyl;
R′ is H, (C₁-C₈)alkyl, (C₃-C₇)cycloalkyl, (C₂-C₈)alkenyl, (C₂-C₈)alkynyl, benzyl, aryl, (C₀-C₄)alkyl)-(C₁-C₆)heterocycloalkyl, (C₀-C₄)alkyl)-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl)-(C₁-C₆)heterocycloalkyl, (C₀-C₄)alkyl)-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl)-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group; and
n is 0 or 1.

32. The process of claim 31, wherein R¹ is —CH═N—N(CH₃)₂, —CH₂NH₂ or an acid salt thereof, and —CH₂—C(═O)—R¹¹ where R¹¹ is cyclopropyl, cyclobutyl, cyclopentyl, 3-cyclopentylpropyl, cyclohexyl, 1-methylcyclohexyl, cycloheptyl, cyclooctyl, cyclodecyl or cyclopentylmethyl.

33. The process of claim 31, wherein the heterocyclic compound of Formula (V) is prepared by the reaction of maleic anhydride with a primary amine having the formula: 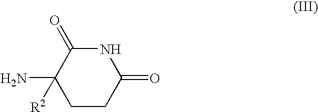
or a salt thereof, where R² is H, F, benzyl, (C₁-C₈)alkyl, (C₂-C₈)alkenyl, or (C₂-C₈)alkynyl.

34. The process of claim 33, wherein the primary amine is 3-aminopiperidine-2,6-dione, 4-alkyl-3-aminopiperidine-2,6-dione or a salt thereof.

35. The process of claim 33, wherein the reaction between maleic anhydride and the primary amine or a salt thereof occurs in the presence of a catalyst comprising a mixture of acetic acid and imidazole.

36. The process of claim 31, wherein R¹ is a —C(R⁷)═N—NR⁸R⁹ group where each of R⁷, R⁸, and R⁹ is independently hydrogen, alkyl, heteroalkyl, aryl, heteroaryl, cycloalkyl or heterocycloalkyl.

37. The process of claim 36 further comprising a third step of reducing the —C(R⁷)═N—NR⁸R⁹ group of Formula (I) to a —CH(R⁷)—NH₂ group by a reducing agent so as to form a compound of Formula (VII): 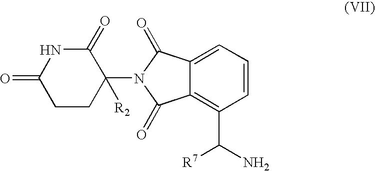
wherein R² is H, F, benzyl, (C₁-C₈)alkyl, (C₂-C₈)alkenyl, or (C₂-C₈)alkynyl.

38. The process of claim 37 wherein the reducing agent is hydrogen with 10% Pd/C.

39. The process of claim 37 further comprising a fourth step of acylating the —CH(R⁷)—NH₂ group of Formula (VII) with an acyl halide having the formula R¹¹—C(═O)-Ha, where Ha is F, Cl, Br or I; and R¹¹ is hydrogen, alkyl, heteroalkyl, aryl, heteroaryl, cycloalkyl or heterocycloalkyl.

40. The process of claim 39 wherein the acyl halide is cyclopropanecarbonyl chloride, cyclobutanecarbonyl chloride, cyclopentanecarbonyl chloride, cyclohexanecarbonyl chloride, cyclopentylacetyl chloride, 1-methylcyclohexanecarbonyl chloride, 3-cyclopentylpropanoyl chloride or cycloheptanecarbonyl chloride.

41. The process of claim 39, wherein the fourth acylating step occurs in the presence of a base catalyst wherein the base catalyst is an organic amine.

42. A process for preparing 4-[(N,N-dimethylhydrazono)methyl]-2-(2,6-dioxopiperidin-3-yl)isoindole-1,3-dione having the formula: 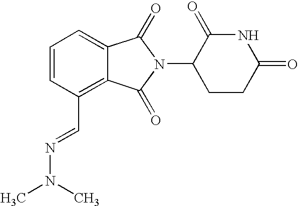
or a pharmaceutically acceptable salt, solvate including a hydrate or polymorph thereof, comprising the steps of:
(1) reacting maleic anhydride with 2-furaldehyde dimethylhydrazone having the formula: 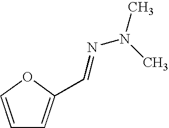
in a first solvent at a first temperature above room temperature to form an isobenzofuran having Formula (X): 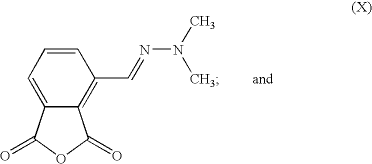
(2) reacting the isobenzofuran with 3-aminopiperidine-2,6-dione hydrochloride in a second solvent at a second temperature above room temperature.

43. The process of claim 42, wherein the first step occurs in the presence of trifluoroacetic acid.

44. The process of claim 42, wherein the first solvent is ethyl acetate.

45. The process of claim 42, wherein the first temperature is between 45° C. and 55° C.

46. The process of claim 42, wherein the second step occurs in the presence of a mixture of acetic acid and imidazole.

47. The process of claim 42, wherein the second solvent is acetonitrile.

48. The process of claim 42, wherein the second temperature is between 75° C. and 85° C.

49. The process of claim 42 further comprising a third step of reducing the —CH═N—N(CH₃)₂ group of the 4-[(N,N-dimethylhydrazono)methyl]-2-(2,6-dioxopiperidin-3-yl)isoindole-1,3-dione to a —CH₂NH₂ group by hydrogen in the presence of 10% Pd/C and methanesulfonic acid to form a mesylate salt of 4-aminomethyl-2-(2,6-dioxopiperidin-3-yl)isoindole-1,3-dione.

50. The process of claim 49, wherein the third reducing step occurs in a mixture of methanol and water and the pressure of hydrogen is between 2.7 and 3.5 bars.

51. The process of claim 49, further comprising reacting the mesylate salt with hydrochloric acid in a molar ratio of 1 to 1 to convert the mesylate salt of 4-aminomethyl-2-(2,6-dioxopiperidin-3-yl)isoindole-1,3-dione to a hydrochloride salt of 4-aminomethyl-2-(2,6-dioxopiperidin-3-yl)isoindole-1,3-dione.

52. The process of claim 51 further comprising a fourth step of acylating the —CH₂—NH₂ group of the 4-aminomethyl-2-(2,6-dioxopiperidin-3-yl)isoindole-1,3-dione hydrochloride with cyclopropanecarbonyl chloride in the presence of N,N-diisopropylethylamine in acetonitrile at a temperature between 0° C. and 20° C. so as to form 4-[(cyclopropanecarbonylamino)methyl]-2-(2,6-dioxopiperidin-3-yl)isoindole-1,3-diones.

53. A process for preparing a compound of Formula (IV): 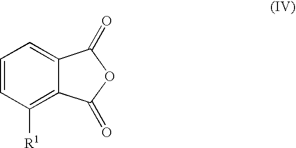
or a pharmaceutically acceptable salt, solvate including a hydrate or polymorph thereof, comprising the step of reacting a furan of Formula (II): 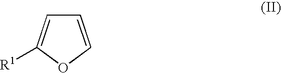
with maleic anhydride in ethyl acetate with the presence of an organic acid; wherein:
R¹ is —(CH₂)ₙ—NH—R′;
R′ is H, (C₁-C₈)alkyl, (C₃-C₇)cycloalkyl, (C₂-C₈)alkenyl, (C₂-C₈)alkynyl, benzyl, aryl, (C₀-C₄)alkyl(C₁-C₆)heterocycloalkyl, (C₀-C₄)alkyl)-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group; and
n is 0 or 1.

54. The process of claim 53, wherein the organic acid is selected from the group consisting of trifluoroacetic acid, 4-(trifluoromethyl)benzoic acid, p-toluenesulfonic acid, methanesulfonic acid, acetic anhydride, and combinations thereof.

55. The process of claim 53, wherein the furan of Formula (II) is 2-furaldehyde dimethylhydrazone.

56. A process for preparing a compound of Formula (I): 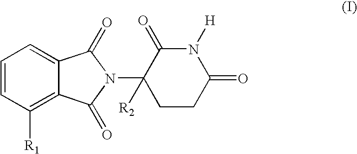
or a pharmaceutically acceptable salt, solvate including a hydrate or polymorph thereof, which comprises the step of reacting a compound of Formula (IV): 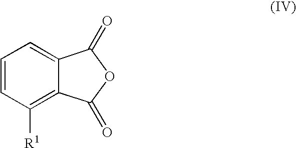
with a primary amine having the formula: 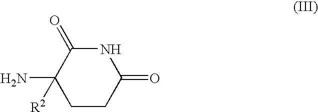
or a salt thereof in the presence of a mixture of acetic acid and imidazole; wherein:
R¹ is —(CH₂)ₙ—NH—R′;
R² is H, F, benzyl, (C₁-C₈)alkyl, (C₂-C₈)alkenyl, or (C₂-C₈)alkynyl;
R′ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl)-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R³′, C(S)NR³R^{3′} or (C₁-C₈)alkyl-O(CO)R⁵;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group; and
n is 0 or 1.

57. The process of claim 56, wherein the compound of Formula (IV) is prepared by reacting maleic anhydride with a furan of Formula (II):

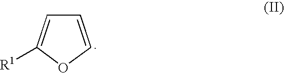

58. The process of claim 56, wherein R¹ Formula (I) is —C(R⁷)═N—NR⁸R⁹ where each of R⁷, R⁸, and R⁹ is independently hydrogen, alkyl, heteroalkyl, aryl, heteroaryl, cycloalkyl or heterocycloalkyl.

59. The process of claim 58, further comprising the step of reducing the —C(R⁷)═N—NR⁸R⁹ group of Formula (I) to a —CH(R⁷)—NH₂ group by a reducing agent so as to form a compound of Formula (VII):

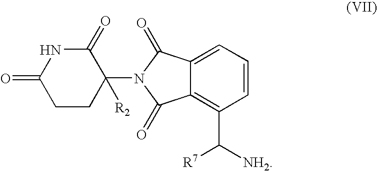

60. The process of claim 59 wherein the reducing agent is hydrogen and the third step occurs in the presence of f 10% Pd/C and methanesulfonic acid.

61. The process of claim 59 further comprising a fourth step of acylating the —CH(R⁷)—NH₂ group of Formula (VII) with an acyl halide having the formula R¹¹—C(═O)-Ha where Ha is F, Cl, Br or I; and R¹¹ is hydrogen, alkyl, heteroalkyl, aryl, heteroaryl, cycloalkyl or heterocycloalkyl.

62. The process of claim 61, wherein the fourth acylating step occurs in the presence of a base catalyst wherein the base catalyst is an organic amine.
